(19) United States
(12) Patent Application Publication
(10) Pub. No.: US 2014/0228579 A1
(43) Pub. Date: Aug. 14, 2014

(54) METHOD FOR THE CATALYTIC REDUCTION OF ACID CHLORIDES AND IMIDOYL CHLORIDES

(71) Applicants: Georgii Nikonov, St. Catharines (CA); Dmitry Gutsulyak, St. Catharines (CA); Sun Hwa Lee, St. Catharines (CA)

(72) Inventors: Georgii Nikonov, St. Catharines (CA); Dmitry Gutsulyak, St. Catharines (CA); Sun Hwa Lee, St. Catharines (CA)

(73) Assignee: Brock Unviersity, St. Catharines (CA)

(21) Appl. No.: 14/023,756

(22) Filed: Sep. 11, 2013

Related U.S. Application Data
(60) Provisional application No. 61/764,754, filed on Feb. 14, 2013.

(51) Int. Cl. C07B 41/06; C07C 51/373; C07D 333/22; C07D 307/48; C07C 45/41; C07D 213/48

(52) U.S. Cl. CPC C07B 41/06; C07C 45/41; C07D 213/48; C07D 333/22; C07D 307/48; C07C 51/373
USPC 546/314; 568/437; 549/70; 549/489; 568/490; 560/177

(57) ABSTRACT

The present application relates to methods for the catalytic reduction of acid chlorides and/or imidoyl chlorides. The methods comprise reacting the acid chloride or imidoyl chloride with a silane reducing agent in the presence of a catalyst such as [Cp(Prⁱ₃P)Ru(NCMe)₂]⁺[PF₆]⁻.

### CROSS-REFERENCE TO RELATED APPLICATIONS

The present application claims the benefit of priority from co-pending U.S. provisional application No. 61/764,754 filed on Feb. 14, 2013, the contents of which are incorporated herein by reference in their entirety.

### FIELD

The present application relates to methods for the catalytic reduction of acid chlorides and/or imidoyl chlorides. In particular, the present application relates to methods for the catalytic reduction of acid chlorides and/or imidoyl chlorides using a silane reducing agent such as dimethylphenylsilane in the presence of a catalyst such as [Cp(Prⁱ₃P)Ru(NCMe)₂]⁺[X]⁻.

### BACKGROUND

Reduction of carbonyl substrates is a fundamental organic reaction allowing for their interconversion (for example, esters to aldehydes) and preparation of a variety of other organic products (for example, alcohols from ketones).¹ Reductions of aldehydes, ketones and imines by silanes have been known for a long period of time, whereas the development of catalytic reduction methods for less reactive substrates has received attention only recently.^{2,3,4,5,6}

Acid chlorides are very reactive compounds but their transformation into aldehydes is known to present a chemoselectivity problem. In addition to the classical Rosenmund reduction by hydrogen, acid chlorides are usually converted to aldehydes by alumohydrides (such as diisobutylaluminium hydride (DIBALH) and borohydrides,^{1,7} tin hydrides,⁸ transition metal hydrides,⁹ or active metals.¹⁰ These methods have issues, for example of cost, toxicity, safety, sensitivity to reaction conditions and/or incompatibility with certain functional groups.

Catalytic reduction by silanes, which may, for example have low toxicity, be air stable and/or have a low cost, is a potential but little studied alternative.^{11,12,13} Earlier studies with Group 9 and 10 metals required harsh conditions,¹² but recently Maleczka et al. reported polymethylhydrosiloxane (PMHS) reduction of a series of aromatic acid chlorides to aldehydes under mild conditions.^{12e} However, the latter method utilizes a Pd catalyst and fails for electron-poor benzoyl chlorides and aliphatic acid chlorides.

Gutsulyak et al. have reported ruthenium-catalyzed hydrosilylation of carbonyls.¹⁴ Methods for the chemoselective hydrosilylation of nitriles^{5c} and pyridines are also known.¹⁵

### SUMMARY

A variety of aromatic, heteroaromatic and alkyl acid chlorides were selectively converted into aldehydes using dimethylphenyl silane (HSiMe₂Ph) as a reducing reagent in the presence of a cationic ruthenium catalyst having the chemical formula [Cp(Prⁱ₃P)Ru(NCMe)₂]⁺[PF₆]⁻. The reactions proceeded under mild conditions and were tolerant of several different functional groups. A variety of aromatic and alkyl imidoyl chlorides were also selectively converted into the corresponding imines using dimethylphenylsilane as a reducing agent in the presence of the catalyst [Cp(Prⁱ₃P)Ru(NCMe)₂]⁺[PF₆]⁻.

Accordingly, the present application includes a method for the catalytic reduction of a compound selected from an acid chloride and an imidoyl chloride, the method comprising reacting the compound with a silane reducing agent in the presence of a catalyst of Formula I:

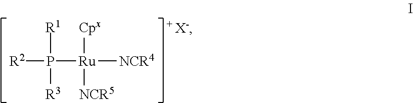

wherein

Cp^{x} is unsubstituted η⁵-cyclopentadienyl or η⁵-cyclopentadienyl substituted with 1 to 5 methyl groups;

R¹, R² and R³ are each independently selected from C₁₋₆alkyl and C₆₋₁₀aryl;

R⁴ and R⁵ are each independently C₁₋₄alkyl; and

X⁻ is a counteranion.

In an embodiment, Cp^{x} is unsubstituted η⁵-cyclopentadienyl.

In another embodiment, the silane reducing agent is selected from dimethylphenylsilane, triethylsilane, methylphenylsilane and triphenylsilane. In a further embodiment, the silane reducing agent is dimethylphenylsilane.

In an embodiment, R¹, R² and R³ are each isopropyl.

In another embodiment, R⁴ and R⁵ are each CH₃.

In a further embodiment, X⁻ is selected from [PF₆]⁻, [BF₄]⁻, [CIO₄⁻], [B[3,5-(CF₃)₂C₆H₃]₄]⁻, [B(C₆F₅)₄]⁻, [Al(OC(CF₃)₃)₄]⁻, a carborane-based counteranion and a non-nucleophilic amide counteranion.

In an embodiment of the present application, the catalyst of Formula I is [Cp(Prⁱ₃P)Ru(NCMe)₂]⁺[PF₆]⁻.

In an embodiment, the compound is an acid chloride having the structure:

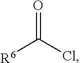

wherein R⁶ is

C₁₋₁₀alkyl, optionally substituted with chloro;

C₆₋₁₄aryl, optionally substituted with halo, nitro or C₁₋₄alkoxy,

heteroaryl; or

—C(O)OR⁷, wherein R⁷ is C₁₋₆alkyl.

In another embodiment, the compound is an acid chloride having the structure:

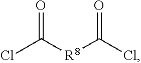

wherein R⁸ is C₁₋₁₀alkylene, C₆₋₁₄arylene or heteroarylene.

In a further embodiment, the compound is an imidoyl chloride having the structure:

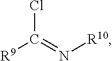

wherein
R⁹ is C₁₋₁₀alkyl or is C₆₋₁₄aryl, optionally substituted with C₁₋₄alkoxy; and
R¹⁰ is C₁₋₆alkyleneC₆₋₁₄aryl or is C₆₋₁₄aryl, optionally substituted with a —C(O)R¹¹ group or a —C(O)OR¹² group, wherein R¹¹ and R¹² are, independently, C₁₋₆alkyl.

In an embodiment, the catalyst is present in an amount of from about 0.2 mol % to about 20 mol %, based on the amount of the compound being reduced.

In an embodiment, the reaction of the compound with the silane reducing agent is carried out in the presence of at least one solvent. In another embodiment, the solvent is selected from chloroform, dichloromethane, acetone and acetonitrile. In a further embodiment, the solvent is selected from chloroform, dichloromethane and acetone.

In an embodiment, the reaction of the compound with the silane reducing agent is further carried out in the presence of a C₁₋₆alkyl cyanide. In another embodiment, the C₁₋₆alkyl cyanide is tBuCN or CH₃CN. In a further embodiment, the C₁₋₆alkyl cyanide is present in an amount of from about 5 mol % to about 250 mol %, based on the amount of the compound being reduced.

In an embodiment, the silane reducing agent is present in an amount of about 1 equivalent to about 2 equivalents, based on the amount of a functional group being reduced.

In an embodiment, the catalyst of Formula I is generated in situ from the reaction of a catalyst precursor of Formula II:

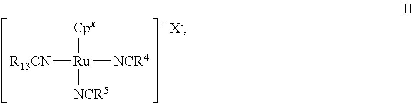

wherein

Cp^{x} is unsubstituted η⁵-cyclopentadienyl or η⁵-cyclopentadienyl substituted with 1 to 5 methyl groups;

R⁴, R⁵ and R¹³ are each independently C₁₋₄alkyl; and

X⁻ is a counteranion,

with a phosphine of Formula III:

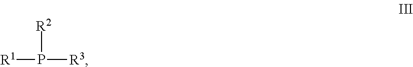

wherein

R¹, R² and R³ are each independently selected from C₁₋₆alkyl and C₆₋₁₀aryl.

In an embodiment, the reaction of the compound with the silane reducing agent in the presence of the catalyst of Formula I is carried out at a temperature of about 20° C. to about 25° C.

### DETAILED DESCRIPTION

### I. Definitions

Unless otherwise indicated, the definitions and embodiments described in this and other sections are intended to be applicable to all embodiments and aspects of the application herein described for which they are suitable as would be understood by a person skilled in the art.

As used in this application, the singular forms “a”, “an” and “the” include plural references unless the content clearly dictates otherwise. For example, an embodiment including “an acid chloride” should be understood to present certain aspects with one acid chloride, or two or more additional acid chlorides.

In embodiments comprising an “additional” or “second” component, such as an additional or second acid chloride, the second component as used herein is chemically different from the other components or first component. A “third” component is different from the other, first, and second components, and further enumerated or “additional” components are similarly different.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “counteranion” as used herein refers to a negatively charged species consisting of a single element, or a negatively charged species consisting of a group of elements connected by ionic and/or covalent bonds that does not react with the cationic portion of the catalyst of Formula I of the present application; i.e. the counteranion is an “innocent” counteranion. For example, counteranions that are coordinating only towards highly electrophilic metal ions (such as the counteranions tetrafluoroborate ([BF₄]⁻), hexafluorophosphate ([PF₆⁻]) and perchlorate ([ClO₄⁻]) and non-nucleophilic amide counteranions) and non-coordinating or weakly-coordinating counteranions (such as [B[3,5-(CF₃)₂C₆H₃]₄]⁻, tetrakis(pentafluorophenyl)borate, [Al(OC(CF₃)₃)₄]⁻ and carborane-based counteranions) are suitable.

The term “carborane-based counteranion” as used herein refers to a negatively charged species that is the conjugate base to a carborane acid. The term “carborane acid” as used herein refers to a polyhedral cluster compound comprising boron and carbon atoms and at least one acidic hydrogen atom. It will be appreciated by a person skilled in the art that carborane-based counteranions such as icosahedral carborane anions, for example CHB₁₁Cl₁₁⁻ are amongst the least coordinating and least basic and most chemically inert anions known.¹⁶ In an embodiment, the carborane-based counteranion is an icosahedral carborane anion such as CHB₁₁R₅Z₆, wherein R is independently selected from H, Me and Cl and Z is independently selected from Cl, Br and I or wherein R and Z are all H or are all Me. In another embodiment, the carborane-based counteranion is selected from CHB₁₁Cl₁₁⁻, HB₁₁(CH₃)₁₁⁻, CHB₁₁H₅Cl₆⁻, CHB₁₁(CH₃)₅Cl₆⁻, CHB₁₁H₁₁⁻, CHB₁₁H₅Br₆⁻, CHB₁₁(CH₃)₅Br₆⁻, CHB₁₁H₅I₆⁻ and CHB₁₁(CH₃)₅I₆⁻. It is an embodiment that the carborane-based counteranion is CHB₁₁Cl₁₁⁻. The selection of a suitable carborane-based counteranion for a particular catalytic reduction and a suitable synthesis and/or source to obtain such a carborane-based counteranion can be made by a person skilled in the art.

The term “non-nucleophilic amide counteranion” as used herein refers to an anionic species that is a poor nucleophile which comprises a negatively charged nitrogen atom. In an embodiment of the present application, the non-nucleophilic amide counteranion is a bis(fluoroalkylsulfonyl)amide such as bis(trifluoromethylsulfonyl)amide (NTf₂⁻). The selection of a suitable non-nucleophilic amide counteranion for a particular catalytic reduction and a suitable synthesis and/or source to obtain such a non-nucleophilic amide counteranion can be made by a person skilled in the art.

The term “alkyl” as used herein, whether it is used alone or as part of another group, means straight or branched chain, saturated alkyl groups. The term C₁₋₆alkyl means an alkyl group having 1, 2, 3, 4, 5 or 6 carbon atoms.

The term “alkylene” as used herein means straight or branched chain, saturated alkylene group, that is, a saturated carbon chain that contains substituents on two of its ends. The term C₁₋₆alkylene means an alkylene group having 1, 2, 3, 4, 5 or 6 carbon atoms.

The term “aryl” as used herein refers to cyclic groups that contain at least one aromatic ring. In an embodiment of the application, the aryl group contains from 6, 9, 10 or 14 atoms, such as phenyl, naphthyl, indanyl or anthracenyl.

The term “arylene” as used herein refers to an aryl group that contains substituents on two of its ends.

The term “alkoxy” as used herein refers to the group “alkyl-O—”. The term “C₁₋₄alkoxy” means an alkoxy group having an alkyl group having 1, 2, 3 or 4 carbon atoms bonded to the oxygen atom of the alkoxy group.

The term “heteroaryl” as used herein means a monocyclic ring or a polycyclic ring system containing 5, 6, 7, 8, 9, 10, 11, 12, 13, 14, 15, 16, 17, 18, 19 or 20 atoms, of which one or more, for example 1 to 8, 1 to 6, 1 to 5, or 1 to 4, of the atoms are a heteromoiety selected from O, S, NH and NC₁₋₆alkyl, with the remaining atoms being C, CH or CH₂, said ring system containing at least one aromatic ring. Examples of heteroaryl groups include, but are not limited to furanyl, thiophenyl, pyrrolyl, imidazolyl, pyrazolyl, triazolyl, oxazolyl, isoxazolyl, thiazolyl, isothiazolyl, oxadiazolyl, tetrazolyl, oxatriazolyl, isoxazinyl, pyridinyl, pyridazinyl, pyrimidinyl, pyrazinyl, triazinyl, benzofuranyl, isobenzofuranyl, benzothiophenyl, indolyl, isoindolyl, quinolinyl, isoquinolinyl, benzodiazinyl, pyridopyridinyl, acridinyl, xanthenyl and the like. In an embodiment of the present application, the heteroaryl is pyridinyl, thiophenyl or furanyl.

The term “heteroarylene” as used herein refers to a heteroaryl group that contains substituents on two of its ends.

The term “halo” as used herein refers to a halogen atom and includes fluoro, chloro, bromo and iodo.

The term “silane reducing agent” as used herein refers to a compound of the formula R″′R″R′Si—H, wherein R′ is selected from H, C₁₋₆alkyl and C₆₋₁₀aryl and R″ and R″′ are independently selected from C₁₋₆alkyl and C₁₋₆aryl.

The term “solvent-free conditions” as used herein means that the reaction is carried out without the addition of a solvent to the reaction mixture or to an individual component thereof but the reaction mixture or an individual component thereof may include small amounts, for example less than about 10, 9, 8, 7, 6, 5, 4, 3, 2, 1, 0.5, 0.1 or 0.01 wt % of one or more solvents.

The term “on bench” as used herein means that the reaction is carried out without the use of strict inert atmosphere conditions such as the use of conventional high-vacuum or nitrogen-line Schlenk techniques or conducting the reaction in an inert atmosphere glove box. For example, in an embodiment the catalyst of Formula I is placed into a vessel such as a vial having an opening closable by a screw cap or a flask having an opening comprising a neck, the vessel is purged by an inert gas such as nitrogen or argon through a septum placed over the opening of the vessel, and the reagents and optionally the at least one solvent added to the vessel through the septum, for example using a cannula or syringe. It will be appreciated by a person skilled in the art that there are other conditions for carrying out a reaction on bench and the selection of suitable conditions for a particular method can be made by a person skilled in the art.

### II. Methods

A variety of aromatic, heteroaromatic and alkyl acid chlorides were observed in the studies of the present application to be selectively converted into aldehydes using a silane reducing reagent in the presence of a cationic ruthenium catalyst. The reactions proceeded under mild conditions and were observed to be tolerant to a variety of functional groups. This new convenient and general method for the chemoselective reduction of acid chlorides to aldehydes will work on bench, is scalable, and/or allows for catalyst recycling. The catalyst has a good shelf life in air and can be assembled prior to use from commercially available materials. A variety of aromatic and alkyl imidoyl chlorides were also selectively converted into the corresponding imines using a silane reducing agent in the presence of the cationic ruthenium catalyst in the present studies.

Accordingly, the present application includes a method for the catalytic reduction of a compound selected from an acid chloride and an imidoyl chloride, the method comprising reacting the compound with a silane reducing agent in the presence of a catalyst of Formula I:

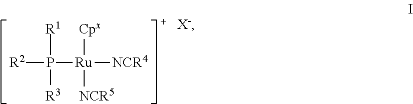

wherein

Cp^{x} is unsubstituted η⁵-cyclopentadienyl or η⁵-cyclopentadienyl substituted with 1 to 5 methyl groups;

R¹, R² and R³ are each independently selected from C₁₋₆alkyl and C₆₋₁₀aryl;

R⁴ and R⁵ are each independently C₁₋₄alkyl; and

X⁻ is a counteranion.

In an embodiment, the silane reducing agent is selected from dimethylphenylsilane, triethylsilane, methylphenylsilane and triphenylsilane. In another embodiment, the silane reducing agent is dimethylphenylsilane.

In an embodiment, Cp^{x} is unsubstituted η⁵-cyclopentadienyl (Cp). In another embodiment, Cp^{x} is η⁵-cyclopentadienyl substituted with 1 to 5 methyl groups. In a further embodiment, Cp^{x} is η⁵-1,2,3,4,5-pentamethylcyclopentadienyl (Cp*). It is an embodiment that Cp^{x} is η⁵-methylcyclopentadienyl (Cp′).

In an embodiment, R¹, R² and R³ are each independently selected from C₁₋₆alkyl and phenyl. In another embodiment, R¹, R² and R³ are each independently selected from C₁₋₆alkyl. In a further embodiment, R¹, R² and R³ are each independently selected from C₁₋₄alkyl. It is an embodiment that R¹ and R² are each isopropyl and R³ is methyl. In an embodiment, R¹, R² and R³ are each methyl. In another embodiment, R¹, R² and R³ are each isopropyl.

In an embodiment, R⁴ and R⁵ are each CH₃.

In an embodiment, X⁻ is selected from [PF₆]⁻, [BF₄]⁻, [ClO₄⁻], [B[3,5-(CF₃)₂C₆H₃]₄]⁻, [B(C₆F₅)₄]⁻, [Al(OC(CF₃)₃)₄]⁻, a carborane-based counteranion and a non-nucleophilic amide counteranion. In another embodiment, X⁻ is [PF₆]⁻.

In an embodiment of the present application, the catalyst of Formula I is [Cp(Prⁱ₃P)Ru(NCMe)₂]⁺[PF₆]⁻.

In another embodiment of the present application, the compound is an acid chloride having the structure:

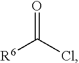

wherein R⁶ is

C₁₋₁₀alkyl, optionally substituted with chloro;

C₆₋₁₄aryl, optionally substituted with halo, nitro or C₁₋₄alkoxy;

heteroaryl; or

—C(O)OR⁷, wherein R⁷ is C₁₋₆alkyl.

It is an embodiment that the acid chloride is selected from:

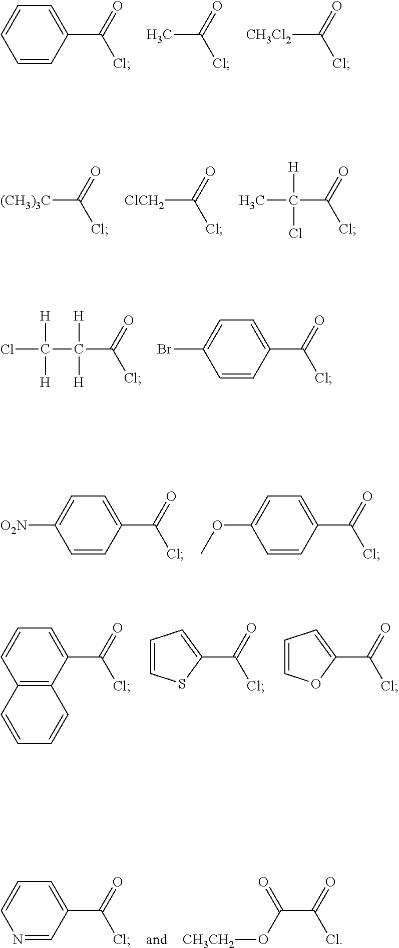

In another embodiment of the present application, the compound is an acid chloride having the structure:

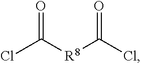

wherein R⁸ is C₁₋₁₀alkylene, C₆₋₁₄arylene or heteroarylene. In a further embodiment, R⁸ is

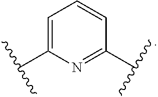 [SMILES: *c1cccc(*)n1]

In an embodiment of the present application, the compound is an imidoyl chloride having the structure:

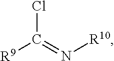

wherein
R⁹ is C₁₋₁₀alkyl or is C₆₋₁₄aryl, optionally substituted with C₁₋₄alkoxy; and
R¹⁰ is C₁₋₆alkyleneC₆₋₁₄aryl or is C₆₋₁₄aryl, optionally substituted with a —C(O)R¹¹ group or a —C(O)OR¹² group, wherein R¹¹ and R¹² are, independently, C₁₋₆alkyl.

In another embodiment, the imidoyl chloride is selected from:

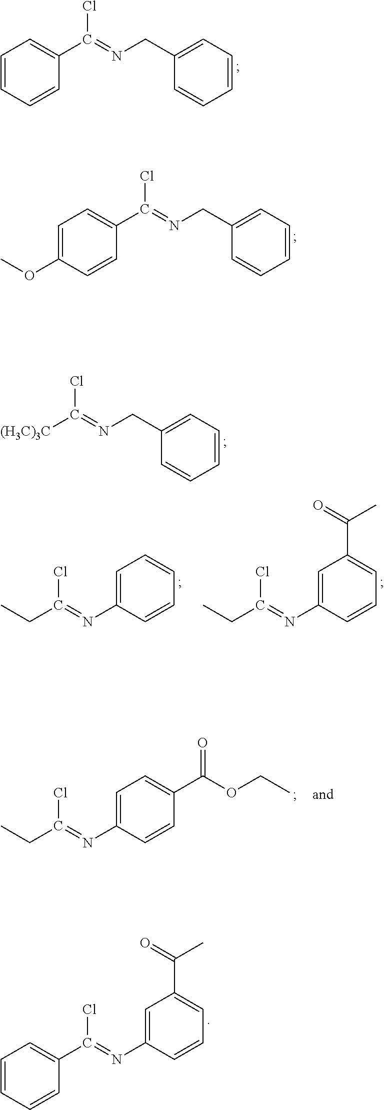

In an embodiment of the present application, the compound is an imidoyl chloride having the structure:

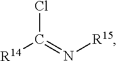

wherein

R¹⁴ is C₆₋₁₄aryl, optionally substituted with chloro, CF₃ or a —C(O)OR¹⁶ group, wherein R¹⁶ is C₁₋₆alkyl; and

R¹⁵ is C₁₋₆alkyl.

In another embodiment, the imidoyl chloride is selected from:

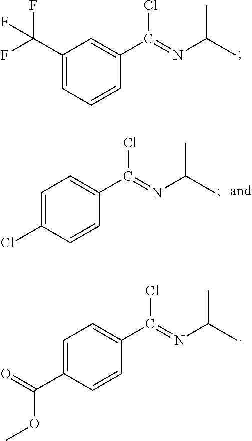 [SMILES: CC(C)/N=C(\Cl)c1cccc(C(F)(F)F)c1 ; CC(C)/N=C(\Cl)c1ccc(Cl)cc1 ; COC(=O)c1ccc(/C(Cl)=N/C(C)C)cc1]

The acid chlorides and imidoyl chlorides for use in the methods of the present application are either commercially available or may be prepared using standard methods known in the art.

It will be appreciated by a person skilled in the art that impurities can deactivate the catalyst of Formula I therefore the lower limit for the amount of the catalyst of Formula I will depend, for example on the purity of the compound being reduced. In an embodiment, the catalyst of Formula I is present in an amount of from about 0.2 mol % to about 20 mol %, about 2 mol % to about 10 mol % or about 4 mol % to about 7 mol %, based on the amount of the compound being reduced. In another embodiment, the catalyst of Formula I is present in an amount of about 5 mol %, based on the amount of the compound being reduced.

In an embodiment, the compound being reduced is a liquid and the reaction of the compound with the silane reducing agent is carried out under solvent-free conditions.

In an alternate embodiment, the reaction of the compound with the silane reducing agent is carried out in the presence of at least one solvent. It will be appreciated by a person skilled in the art that the catalyst of Formula I is a cationic catalyst therefore the use of a solvent or a mixture of solvents that is polar would be useful. In an embodiment, the solvent is an inert organic solvent that does not interfere with the reaction. In another embodiment, the solvent is a chlorinated solvent such as chlorobenzene, chloroform or dichloromethane, a ketone such as acetone, a nitrile such as acetonitrile or an amide such as dimethylformamide (DMF) or N-methyl-2-pyrrolidone. In an embodiment, the solvent is selected from chloroform, dichloromethane, acetone and acetonitrile. In another embodiment, the solvent is selected from chloroform, dichloromethane and acetone. In a further embodiment, the solvent is chloroform or dichloromethane. It is an embodiment that the solvent is acetone. In an embodiment, the solvent is acetonitrile.

In some embodiments of the present application, for example where the solvent is chloroform, dichloromethane or acetone, carrying out the method for the catalytic reduction of the compound selected from an acid chloride and an imidoyl chloride in the presence of an alkyl cyanide such as t-BuCN or CH₃CN will improve the yield of the reaction. Accordingly, in an embodiment of the present application, the reaction of the compound with the silane reducing agent is further carried out in the presence of a C₁₋₆alkyl cyanide. In an embodiment, the C₁₋₆alkyl cyanide is t-BuCN or CH₃CN. The hydrosilylation of acetonitrile has been observed to occur as a minor reaction during the reaction of an acid chloride with a silane reducing agent in the presence of a catalyst of Formula I and acetonitrile. It will therefore be appreciated by a person skilled in the art that the use of a C₁₋₆alkyl cyanide such as t-BuCN that is known to be more inert than acetonitrile is useful for a selective catalytic reduction of an acid chloride in the methods of the present application. In an embodiment, the selection of a suitable C₁₋₆alkyl cyanide depends on the cost of a particular C₁₋₆alkyl cyanide. For example, it is known that acetonitrile is generally less expensive than t-BuCN.

In another embodiment, the C₁₋₆alkyl cyanide is present in an amount of at least about 5 mol % based on the amount of the compound being reduced. In a further embodiment, the C₁₋₆alkyl cyanide is present in an amount of from about 5 mol % to about 250 mol % based on the amount of the compound being reduced. It is an embodiment that the compound is an acid chloride and the C₁₋₆alkyl cyanide is present in an amount of at least about 5 mol % based on the amount of the compound being reduced. In another embodiment, the compound is an acid chloride and the C₁₋₆alkyl cyanide is present in an amount of from about 5 mol % to about 250 mol % based on the amount of the acid chloride being reduced. In another embodiment, the compound is an acid chloride, the C₁₋₆alkyl cyanide is CH₃CN and the CH₃CN is present in an amount of at least about 5 mol %, based on the amount of the acid chloride being reduced. In another embodiment, the CH₃CN is present in an amount of about 5 mol % to about 250 mol %, about 150 mol % to about 250 mol % or about 200 mol % based on the amount of the acid chloride being reduced. In a further embodiment, the compound is an acid chloride, the C₁₋₆alkyl cyanide is t-BuCN and the t-BuCN is present in an amount of at least about 5 mol %, about 5 mol % to about 15 mol % or about 10 mol % based on the amount of the acid chloride being reduced. In an embodiment, the compound is an imidoyl chloride and the C₁₋₆alkyl cyanide is present in an amount of at least about 5 mol % or about 5 mol % to about 250 mol % based on the amount of the imidoyl chloride being reduced. In an embodiment, the compound is an imidoyl chloride, the C₁₋₆alkyl cyanide is CH₃CN and the CH₃CN is present in an amount of at least about 5 mol % or about 5 mol % to about 250 mol % based on the amount of the imidoyl chloride being reduced. In another embodiment, the compound is an imidoyl chloride, the C₁₋₆alkyl cyanide is t-BuCN and the t-BuCN is present in an amount of at least about 5 mol %, about 10 mol % to about 30 mol % or about 20 mol % to about 25 mol % based on the amount of the imidoyl chloride being reduced.

A person skilled in the art would readily appreciate that the amount of the silane reducing agent will vary, for example, depending on the amount of water present in a solvent used for the reaction and/or the reaction of the silane with a C₁₋₆alkyl cyanide and/or with a solvent, for example, the hydrosilylation of a solvent such as acetone or the chlorination of the silane by a chlorinated solvent such as chloroform used in the reaction and that, for example, an increased amount of silane is added to the reaction mixture if the solvent comprises an increased amount of water and/or the if silane reacts with a C₁₋₆alkyl cyanide and/or with a solvent that is used in the reaction. In an embodiment, the silane reducing agent is present in an amount of about 1 equivalent to about 2 equivalents based on the amount of a functional group being reduced. In another embodiment, the silane reducing agent is present in an amount of about 1.5 equivalents based on the amount of a functional group being reduced.

In an embodiment, the catalyst of Formula I is generated in situ from the reaction of a catalyst precursor of Formula II:

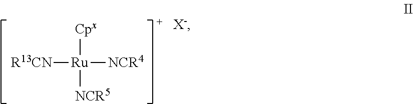

wherein

Cp^{x} is unsubstituted η⁵-cyclopentadienyl or η⁵-cyclopentadienyl substituted with 1 to 5 methyl groups;

R⁴, R⁵ and R¹³ are each independently C₁₋₄alkyl; and

X⁻ is a counteranion,

with a phosphine of Formula III:

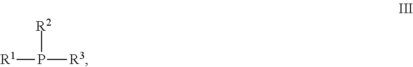

wherein

R¹, R² and R³ are each independently selected from C₁₋₆alkyl and C₆₋₁₀aryl.

In another embodiment, the method further comprises recycling the catalyst for use in a further reaction. For example, the catalyst is separated from the products by precipitation with a suitable non-polar inert solvent such as hexane or benzene, followed by isolating the precipitated catalyst, for example, by filtration, such as by vacuum filtration, and optionally drying to remove at least a portion of residual solvent, if any, remaining on the catalyst after the isolation.

In some embodiments of the present application, the reaction of an acid chloride with a silane reducing agent in the presence of a catalyst of Formula I results in the conversion of at least a portion of the acid chloride to the corresponding silyl enol. It will be appreciated by a person skilled in the art that a silyl enol can be converted to a corresponding aldehyde by methods known in the art, for example using an aqueous work-up as reported by Novice et al.¹⁸

In some embodiments of the present application, the imine prepared from the reaction of an imidoyl chloride with a silane reducing agent in the presence of a catalyst of Formula I is hydrolyzed under conditions to obtain the corresponding aldehyde. For example, a mixture of water and a suitable acid such as hydrochloric acid is added to a product mixture comprising the imine.

It will be appreciated to a person skilled in the art that the catalyst of Formula I is reasonably stable in air. Accordingly, in an embodiment of the present application, the reaction is carried out on bench.

The temperature at which the reaction of the compound with the silane reducing agent in the presence of the catalyst of Formula I is carried out can, for example have an effect on the selectivity of the reaction. For example, higher temperatures are expected to result in a lower selectivity. The selection of a suitable temperature can be made by a person skilled in the art. In an embodiment, the reaction of the compound with the silane reducing agent in the presence of the catalyst of Formula I is carried out at a temperature of from about 0° C. to about 60° C., about 15° C. to about 30° C., about 20° C. to about 25° C., or about room temperature.

### EXAMPLES

### General Experimental Details

All manipulations were carried out using conventional high-vacuum or nitrogen-line Schlenk techniques. NMR spectra were recorded on Bruker (¹H, 300 MHz; ¹³C, 75.4 MHz) and/or Bruker (¹H, 600 MHz; ¹³C, 150.8 MHz) spectrometers. All chemicals used in the reactions were purchased from Sigma-Aldrich, apart from HSiMe₂Ph which was purchased from Gelest. These reagents were used without purification. NMR solvents were obtained from Cambridge Isotope Laboratories. CDCl₃ was dried over CaH₂ and acetone-d₆ was dried over molecular sieves (3 Å). Other solvents were dried by distillation from appropriate drying agents or using a Grubbs-type solvent purification system.

[Cp(Prⁱ₃P)Ru(NCMe)₂]⁺[PF₆]⁻ was prepared according to a literature procedure:¹⁷ To a stirred yellow solution of [CpRu(CH₃CN)₃]⁺[PF₆]⁻ (0.500 g, 1.15 mmol) in CH₃CN (20 mL) was added PPrⁱ₃ (0.22 mL, 1.15 mmol) via syringe. The resulting solution was stirred for 3 h at ambient temperature. All volatiles were then removed under vacuum and the residue was washed with ether (2×20 mL) and hexane (3×10 mL). The product was dried under vacuum affording [Cp(Prⁱ₃P)Ru(NCMe)₂]⁺[PF₆]⁻ as a yellow solid. Yield: 0.570 g (90%).

### Example 1

### Catalytic Reduction of Acid Chlorides

### I. General Procedure for Reactions in NMR Tubes

In a representative procedure, to a solution of acid chloride such as 4-BrC₆H₄COCl (0.043 g, 0.20 mmol), HSiMe₂Ph (0.030 mL, 0.20 mmol), internal standard (tetramethylsilane (TMS) or Cp₂Fe), and t-BuCN (0.002-0.004 mL, 10-20 mol %) in acetone-d₆ (0.6 mL) was added [Cp(Prⁱ₃P)Ru(NCMe)₂]⁺[PF₆]⁻ (0.005 g, 5 mol %). The resulting mixture was mixed at room temperature and the progress of the reaction was monitored by NMR spectroscopy. The order of mixing the reagents other than the catalyst can differ as the reagents have not been observed to react with each other in the absence of a catalyst. However, it is useful to add the catalyst as the last reagent because addition of the catalyst before the nitrile has been observed to result in the deactivation of the catalyst.

### II. Discussion

Complex [Cp(Prⁱ₃P)Ru(NCMe)₂]⁺[PF₆]⁻ catalyzed the chemoselective reduction of a variety of acid chlorides to the corresponding aldehydes using HSiMe₂Ph as the reducing agent (Table 1). No reduction was observed in the absence of the catalyst. The reaction proceeds in various solvents (CH₂Cl₂, acetone, acetonitrile), with acetone being optimum. For example, the reaction time for the reduction of benzoyl chloride in acetone in the presence of CH₃CN (about 3 hours) was observed to be shorter than in other solvents (about 24 hours) under the same conditions (5 mol % catalyst, 200 mol % CH₃CN, room temperature). Depending on the solvent, the addition of t-BuCN (10-20 mol %), was useful for the success of this reaction, as this nitrile stabilizes the catalyst without concomitant nitrile hydrosilylation.¹⁵ For example, aldehyde was only observed to form to a minor extent (<5%) in chlorinated solvents in the absence of nitrile. The reaction in acetone without t-BuCN was also observed to give only small amounts of aldehyde (<10% for the reduction of benzoyl chloride). Reduction in acetonitrile was carried out in the absence of t-BuCN. This reaction was compromised, however, by some concomitant hydrosilylation of the solvent.

Under these conditions, PhCOCl can be quantitatively reduced to PhC(O)H within only 1 h at room temperature (Table 1, entry 1). It was also observed that acyl chlorides (entries 2-8) could be reduced in addition to the aromatic derivatives (entries 1 and 10-13). Chlorine substituents in the α- and β-positions were tolerated and the corresponding aldehydes were obtained with high selectivity (entries 5-7), but only traces of aldehyde were observed for the more reactive bromide derivative BrCH₂CH₂COCl (entry 8). The less substituted substrates (entries 2 and 3) reacted faster than a sterically loaded substrate (entry 4). The reduction of some substrates containing α-protons resulted in the formation of a mixture of the target aldehydes as well as silyl enols (e.g., entries 2 and 7). The latter compounds can be easily converted into aldehydes, for example by a simple aqueous work-up.¹⁸

Chemoselectivity was lost, however, in the case of a conjugated acid chloride, PhCH═CHCOCl (entry 9), as a mixture of the corresponding aldehyde (13%) and the product of the formal silane 1,4-addition to the aldehyde (57%) was observed. The hydrosilylation of PhCH═CHCHO under similar conditions was very sluggish, which suggests, while not wishing to be limited by theory, that PhCH₂CH═CHOSiMe₂Ph does not stem from the direct addition of silane to aldehyde.

Reduction of aromatic acid chlorides with electron-withdrawing groups (entries 10 and 11) proceeded as did the reduction of electro-neutral substrates (entries 1 and 13). The reactive nitro-group was tolerated (entry 11). In contrast, the reduction of an electron-rich substrate, with a donating MeO-group(entry 12), was much slower and some loss of chemoselectivity occurred, leading to a mixture of the target aldehyde (83%) and its hydrosilylation product MeOC₆H₄CH₂OSiMe₂Ph (17%).

A similar reactivity pattern was observed in the reactions of heteroaromatic acid chlorides. The electron-poor 2,6-pyridinedicarbonyl chloride was converted into a mixture of the corresponding mono- and bis-pyridinecarboxaldehydes, with the 2,6-bis-pyridinecarboxaldehyde becoming the predominant product (˜80%) only after 3 h at room temperature (entry 14). In contrast, the reduction of electron-rich furan and thiophene derivatives went to completion after 24 h (entries 15 and 16). Surprisingly, the course of reduction of pyridine substrates was sensitive to the position of the COCl group in the ring, as the reaction of a 3-substituted derivative gave only traces of the aldehyde product (entry 17). However, this reaction might have been compromised by the presence of an excess amount of HCl in the reaction mixture. The ester functionality in the reduction of EtOOC—COCl was tolerated (entry 18).

### Example 2

### Selectivity of Catalytic Reaction of Acid Chlorides

### I. General Reaction Procedure

In a general procedure, to a solution of acid chloride and substrate 2 in acetone-d₆ was added 1.5 equiv. of HSiMe₂Ph, 2 equiv. of CH₃CN and 5 mol % of [Cp(Prⁱ₃P)Ru(NCMe)₂]⁺[PF₆]⁻.

### II. Discussion

To establish further the selectivity of this reduction method, the reaction of 4-bromobenzoyl chloride with HSiMe₂Ph was studied in the presence of other potentially reactive compounds, such as alkenes, alkynes, esters and benzoic acid. The results for the alkene, alkynes and ester studied are listed in Table 2. The presence of hex-1-ene or ethyl ester did not hamper the course of reduction of 4-Br(C₆H₄)COCl (entries 1 and 2). On the other hand, the hydrosilylation of an internal triple bond C≡C of alkyne proceeded as fast as the reduction of the COCl group, and a mixture of EtOH═C(SiMe₂Ph)Et (50%) and 4-Br(C₆H₄)CHO (50%) was obtained in the presence of hex-3-yne (entry 3). Surprisingly, no hydrosilylation of the terminal triple bond of PhC≡CH was observed. However, the latter compound poisons the catalyst, and the reduction of 4-Br(C₆H₄)COCl stops only at 50% conversion (entry 4). Benzoic acid was observed to react with HSiMe₂Ph under the conditions used for the reaction but was not observed to deactivate the catalyst.

### Example 3

### Preparative Scale Reduction of Acid Chlorides with Catalyst Recycling

### I. Representative Example

To a solution of 4-Br(C₆H₄)C(O)Cl (0.500 g, 2.28 mmol), HSiMe₂Ph (0.400 mL, 2.61 mmol), and t-BuCN (0.025 mL, 10 mol %) in CH₂Cl₂ or acetone (30 mL) was added [Cp(Prⁱ₃P)Ru(NCMe)₂]⁺[PF₆]⁻ (0.065 g, 5 mol %). If the solvent was not dry enough, more silane was added to the reaction mixture. The resulting mixture was stirred at room temperature. Full conversion of the acid chloride occurred within 3 h (in acetone) or 1 day (in CH₂Cl₂). To the resulting mixture was added hexane (30 mL) and the solution was concentrated to 5 mL under vacuum. The products were extracted with hexane (3×10 mL). The remaining catalyst was used again (4 times) with the same amounts of the starting reagents. The aldehyde was recrystallized each time from hexane solutions at −80° C. Yields 70% for the first two reactions in CH₂Cl₂ (for both reactions, full conversion of the starting acid chloride was achieved after 1 day at room temperature); 85-95% for the following three reactions in acetone (for all three reactions it took 3 h for the full conversion of the starting acid chloride at room temperature).

### II. Discussion

To explore the scale-up of the present reduction method, a reaction with a representative acid chloride was performed on a preparative scale. Thus, the reaction of 4-Br(C₆H₄)C(O)Cl (0.5 g) with HSiMe₂Ph in the presence of [Cp(Prⁱ₃P)Ru(NCMe)₂]⁺[PF₆]⁻ (5 mol %) and t-BuCN (10 mol %) afforded the corresponding aldehyde in 80% isolated yield after recrystallization from hexanes. This procedure does not require a special set-up and can be performed on the bench in a flask pre-flushed with inert gas. The catalyst is recyclable and can be separated from products by precipitation with hexane. Although there is a slow decomposition of the catalyst during the reaction, most of it can be recycled at least five times without any significant decrease in activity. For example, the reaction time was observed to be about the same for at least five cycles.

### Example 4

### Mechanistic Studies of Acid Chloride Reduction

For the hydrosilylation of pyridine and nitriles catalyzed by [Cp(Prⁱ₃P)Ru(NCMe)₂]⁺[PF₆]⁻, an ionic mechanism, based on nucleophilic abstraction of a silylium ion by the nitrogen donor has been proposed.^{[5c, 15]} Although acid chlorides are weaker nucleophiles, the observation that their reduction proceeds at rates much slower than the hydrosilylation of nitriles was surprising,^{[5c]} but nevertheless aldehydes can be obtained in the presence of acetonitrile. To address this paradox, the possibility that the chloride may be reduced by the initial product of nitrile hydrosilylation, the imine RHC═NSiMe₂Ph was first considered. However, the latter was found to react with acid chlorides to give acyl imines R′C(O)N═CHR, which then undergo reduction of the imine group. Another possibility, a radical mechanism, was then ruled out as the addition of stoichiometric amounts of TEMPO (2,2,6,6-tetramethylpiperidine 1-oxyl), a known radical scavenger, did not affect the rate of reduction of t-BuCOCl.

When a 1:1:1 reaction of 4-Br(C₆H₄)COCl, HSiMe₂Ph, and [Cp(Prⁱ₃P)Ru(NCMe)₂]⁺[PF₆]⁻ was followed by VT NMR, a noticeable reaction was observed at −25° C. with the formation of the known complex {Cp[(Prⁱ)₃P]Ru(NCCH₃)(η²-HSiMe₂Ph)]⁺.¹⁴ However, further gentle increase of temperature to 0° C. resulted in the fast production of aldehyde and no further intermediates were detectable.

### Example 5

### Reduction of Imidoyl Chlorides

### I. Synthesis of Secondary Amides and Imidoyl Chlorides

### PhCONHCH₂Ph

To a solution of benzyl amine (20 mmol, 2.2 mL) in CH₂Cl₂ (30 mL) was added benzoyl chloride (20 mmol, 2.8 mL) and the reaction mixture was stirred overnight at ambient temperature. The mixture was then filtered and the solvent of filtrate was removed in vacuum. The product was washed with hexane (10 mL). Compound N-benzylbenzamide was obtained as a white powder after removal of hexane in vacuum. Yield 3.7 g (88%).

### PhCCl═NCH₂Ph

To a solution of N-benzylbenzamide in CH₂Cl₂ (15 mL) was added 1.1 eq. of distilled Cl₂SO and the reaction mixture was stirred overnight at 70° C. Solvent was then removed in vacuum and the product was distilled under vacuum. Compound PhCCl═NCH₂Ph was obtained as an orange-yellow oil. Yield 1.35 g (63%).

¹H NMR (CH₂Cl₂): δ 7.10-7.91 (m, 10, PhCCl═NCH₂Ph), 4.76 (s, 2, PhCCl═NCH₂Ph).

### 4-MeOC₆H₄CONHCH₂Ph

To a solution of benzyl amine (5 mmol, 0.84 mL) in CH₂Cl₂ (30 mL) was added 4-methoxybenzoyl chloride (5 mmol, 0.85 mL) and the reaction mixture was stirred overnight at ambient temperature. The mixture was then filtered and the solvent of filtrate was removed in vacuum. The product was washed with hexane (10 mL). Compound N-benzyl-4-methoxybenzamide was obtained as a white powder after removal of hexane in vacuum. Yield 0.97 g (85%).

### 4-MeOC₆H₄CCl═NCH₂Ph

To a solution of N-benzyl-4-methoxybenzamide in CH₂Cl₂ (15 mL) was added 1.1 eq. of distilled Cl₂SO and the reaction mixture was stirred overnight at 70° C. Solvent was then removed in vacuum and the product was distilled under vacuum. Compound 4-MeOC₆H₄CCl═NCH₂Ph was obtained as a yellow oil. Yield 0.60 g (64%).

¹H NMR (CH₂Cl₂): δ 7.93-7.96 (d, 2,4-MeOPhCCl═NCH₂Ph), 7.29-7.31 (d, 2,4-MeOPhCCl═NCH₂Ph(o)), 7.19-7.24 (t, 2, 4-MeOPhCCl═NCH₂Ph(m)), 7.11-7.16 (t, 1,4-MeOPhCCl═NCH₂Ph(p)), 6.79-6.82 (d, 2,4-MeOPhCCl═NCH₂Ph), 4.78 (s, 2,4-MeOPhCCl═NCH₂Ph), 3.71 (s, 3, 4-MeOPhCCl═NCH₂Ph).

### ^{t}BuCONHCH₂Ph

To a solution of benzyl amine (5 mmol, 0.84 mL) in CH₂Cl₂ (30 mL) was added trimethylacetyl chloride (5 mmol, 0.60 mL) and the reaction mixture was stirred overnight at ambient temperature. The mixture was then filtered and the solvent of filtrate was removed in vacuum. The product was washed with hexane (10 mL). Compound ^{t}BuCCONHCH₂Ph was obtained as a white powder after removal of hexane in vacuum. Yield 0.60 g (70%).

### ^{t}BuCCl═NCH₂Ph

To a solution of ^{t}BuCCONHCH₂Ph in CH₂Cl₂ (15 mL) was added 1.1 eq. of distilled Cl₂SO and the reaction mixture was stirred overnight at 70° C. Solvent was then removed in vacuum and the product was distilled under vacuum. Compound ^{t}BuCCl═NCH₂Ph was obtained as a white oil. Yield 1.2 g (60%).

¹H NMR (CH₂Cl₂): δ 7.18-7.20 (m, 4, ^{t}BuCCl═NCH₂Ph), 7.09-7.13 (m, 1, ^{t}BuCCl═NCH₂Ph(p)), 4.55 (s, 2, ^{t}BuCCl═NCH₂Ph), 1.17 (s, 9, (m, 1, ^{t}BuCCl═NCH₂Ph).

### CH₃CH₂CONHPh

To a solution of aniline (7.5 mmol, 0.70 mL) in CH₂Cl₂ (30 mL) was added propionyl chloride (7.5 mmol, 0.70 mL) and the reaction mixture was stirred overnight at ambient temperature. The mixture was then filtered and the solvent of filtrate was removed in vacuum. The product was washed with hexane (10 mL). Compound N-phenylpropionamide was obtained as a light yellow powder after removal of hexane in vacuum. Yield 0.60 g (47%).

### CH₃CH₂CCl═NPh

To a solution of CH₃CH₂CONHPh in CH₂Cl₂ was added 1 eq. of PCl₅ and the reaction mixture was stirred for 1 h at room temperature. Solvent was then removed in vacuum and compound CH₃CH₂CCl═NPh was obtained as a white oil.

¹H NMR (CH₂Cl₂): δ 7.18-7.23 (t, 2, CH₃CH₂CCl═NPh(m)), 6.98-7.03 (t, 1, CH₃CH₂CCl═NPh(p)), 6.70-6.72 (t, 2, CH₃CH₂CCl═NPh(o)), 2.61-2.68 (q, 2, CH₃CH₂CCl═NPh), 1.13-1.18 (t, 3, CH₃CH₂CCl═NPh).

### CH₃CH₂CONH(4-C₆H₄COCH₃)

To a solution of 3-aminoacetophenone (7.5 mmol, 1.01 mg) in CH₂Cl₂ (30 mL) was added propionyl chloride (7.5 mmol, 0.70 mL) and the reaction mixture was stirred overnight at ambient temperature. The mixture was then filtered and the solvent of filtrate was removed in vacuum. The product was washed with hexane (10 mL). Compound N-(3-acetylphenyl)propionamide was obtained as a light yellow powder after removal of hexane in vacuum. Yield 0.69 g (48%).

### CH₃CH₂CCl═N(4-C₆H₄COCH₃)

To a solution of CH₃CH₂CONH(4-C₆H₄COCH₃) in CH₂Cl₂ was added 1 eq. of PCl₅ and the reaction mixture was stirred for 1 h at room temperature. Solvent was then removed in vacuum and compound CH₃CH₂CCl═N(4-C₆H₄COCH₃) was obtained as a white oil.

¹H NMR (CH₂Cl₂): δ 7.58-7.60 (d, 1, CH₃CH₂CCl═NPhCOCH₃), 7.29-7.34 (t, 1, CH₃CH₂CCl═NPhCOCH₃), 7.28 (s, 1, CH₃CH₂CCl═NPhCOCH₃), 6.91-6.94 (d, 1, CH₃CH₂CCl═NPhCOCH₃), 2.64-2.71 (q, 2, CH₃CH₂CCl═NPhCOCH₃), 2.43 (s, 3, CH₃CH₂CCl═NPhCOCH₃), 1.15-1.20 (d, 1, CH₃CH₂CCl═NPhCOCH₃).

### CH₃CH₂CONH(4-C₆H₄COOCH₂CH₃)

To a solution of ethyl-4-aminobenzoate (7.5 mmol, 1.24 mg) in CH₂Cl₂ (30 mL) was added propionyl chloride (7.5 mmol, 0.70 mL) and the reaction mixture was stirred overnight at ambient temperature. The mixture was then filtered and the solvent of filtrate was removed in vacuum. The product was washed with hexane (10 mL). Compound ethyl 4-propionamidobenzoate was obtained as a white powder after removal of hexane in vacuum. Yield 0.74 g (45%).

### CH₃CH₂CCl═N(4-C₆H₄COOCH₂CH₃)

To a solution of CH₃CH₂CONH(4-C₆H₄COOCH₂CH₃) in CH₂Cl₂ was added 1 eq. of PCl₅ and the reaction mixture was stirred for 1 h at room temperature. Solvent was then removed in vacuum and compound CH₃CH₂CCl═NPhCOOCH₂CH₃ was obtained as a pale yellow oil.

¹H NMR (CH₂Cl₂): δ 7.86-7.89 (d, 2, NPhCOOCH₂CH₃), 6.74-6.77 (d, 2, NPhCOOCH₂CH₃), 4.15-4.22 (q, 2, NPhCOOCH₂CH₃), 2.64-2.71 (q, 2, CH₃CH₂CCl═N), 1.20-1.24 (t, 3, NPhCOOCH₂CH₃), 1.14-1.19 (t, 3, CH₃CH₂CCl═N).

### N-benzylthiophene-2-carboxamide

To a solution of benzyl amine (5 mmol, 0.84 mL) in CH₂Cl₂ (30 mL) was added thiophene-2-carbonyl chloride (5 mmol, 0.73 mL) and the reaction mixture was stirred overnight at ambient temperature. The mixture was then filtered and the solvent of filtrate was removed in vacuum. The product was washed with hexane (10 mL). Compound N-benzylthiophene-2-carboxamide was obtained as a white powder after removal of hexane in vacuum. Yield 0.70 g (69%).

### N-benzylthiophene-2-carbimidoyl chloride

To a solution of N-benzylthiophene-2-carboxamide in CH₂Cl₂ was added 1 eq. of PCl₅ and the reaction mixture was stirred for 1 h at room temperature. Solvent was then removed in vacuum and compound N-benzylthiophene-2-carbimidoyl chloride was obtained.

¹H NMR (CH₂Cl₂): δ 7.57-7.58 (d, 1, C₄H₃SCCl), 7.35-7.36 (d, 1, C₄H₃SCCl), 7.12-7.27 (m, 5, C₄H₃SCCl═NCH₂Ph), 6.93-6.96 (t, 1, C₄H₃SCCl), 4.72 (s, 2, C₄H₃SCCl═NCH₂Ph).

### N-benzylfuran-2-carboxamide

To a solution of benzyl amine (5 mmol, 0.84 mL) in CH₂Cl₂ (30 mL) was added 2-furoyl chloride (5 mmol, 0.65 mL) and the reaction mixture was stirred overnight at ambient temperature. The mixture was then filtered and the solvent of filtrate was removed in vacuum. The product was washed with hexane (10 mL). Compound N-benzylfuran-2-carboxamide was obtained as a white powder after removal of hexane in vacuum. Yield 0.64 g (68%).

### N-benzylfuran-2-carbimidoyl chloride

To a solution of N-benzylfuran-2-carboxamide in CH₂Cl₂ was added 1 eq. of PCl₅ and the reaction mixture was stirred for 1 h at room temperature. Solvent was then removed in vacuum and compound N-benzylthiophene-2-carbimidoyl chloride was obtained.

¹H NMR (CH₂Cl₂): δ 7.45 (d, 1, C₄H₃OCCl), 7.12-7.27 (m, 5, C₄H₃OCCl═NCH₂Ph), 7.00-7.01 (d, 1, C₄H₃OCCl), 6.39-6.41 (dd, 1, C₄H₃OCCl), 4.74 (s, 2, C₄H₃OCCl═NCH₂Ph).

### N-benzylnicotinamide

To a solution of benzyl amine (5 mmol, 0.84 mL) in CH₂Cl₂ (30 mL) was added nicotinoyl chloride (5 mmol, 0.89 mg) and Et₃N (10 mmol, 1.02 mL). The reaction mixture was stirred overnight at ambient temperature. The mixture was then filtered and the solvent of filtrate was removed in vacuum. The crude product was extracted with Et₂O (20 mL×2). Compound N-benzylnicotinamide was obtained as a white powder after removal of Et₂O in vacuum. Yield 0.60 g (57%).

### N-benzylnicotinimidoyl chloride

To a solution of N-benzylnicotinamide in CH₂Cl₂ was added 1 eq. of PCl₅ and the reaction mixture was stirred for 1 h at room temperature. Solvent was then removed in vacuum and compound N-benzylnicotinimidoyl chloride was obtained.

¹H NMR (CH₂Cl₂): δ 9.11-9.12 (d, 1, C₅H(2)₄NCCl), 8.57-8.59 (dd, 1, C₅H(6)₄NCCl), 8.37-8.40 (d, 1, C₅H(4)₄NCCl), 7.41-7.45 (d, 1, C₅H(5)₄NCCl), 7.19-7.29 (m, 4, CCl═NCH₂Ph), 7.12-7.17 (t, 1, CCl═NCH₂Ph), 4.80 (s, 2, CCl═NCH₂Ph).

### PhCH═CHCONHCH₂Ph

To a solution of benzyl amine (5 mmol, 0.84 mL) in CH₂Cl₂ (30 mL) was added cinnamoyl chloride (5 mmol, 0.83 mg) and the reaction mixture was stirred overnight at ambient temperature. The mixture was then filtered and the solvent of filtrate was removed in vacuum. The product was washed with hexane (10 mL). Compound N-benzylfuran-2-carboxamide was obtained as a white powder after removal of hexane in vacuum. Yield 0.90 g (81%).

### PhCH═CHCCl═NCH₂Ph

To a solution of PhCH═CHCONHCH₂Ph in CH₂Cl₂ was added 1 eq. of PCl₅ and the reaction mixture was stirred for 1 h at room temperature. Solvent was then removed in vacuum and compound PhCH═CHCCl═NCH₂Ph was obtained.

¹H NMR (CH₂Cl₂): δ 7.59-7.75 (q, 2, PhCH═CHCCl), 7.19-7.53 (m, 10, PhCH═CHCCl═NCH₂Ph), 4.83 (s, 2, PhCH═CHCCl═NCH₂Ph).

### CH₃CH₂CONH(4-C₆H₄CN)

To a solution of 4-aminobenzonitrile (7.5 mmol, 0.89 mg) in CH₂Cl₂ (30 mL) was added propionyl chloride (7.5 mmol, 0.70 mL) and the reaction mixture was stirred overnight at ambient temperature. The mixture was then filtered and the solvent of filtrate was removed in vacuum. The product was washed with hexane (10 mL). Compound N-(4-cyanophenyl)propionamide was obtained as a white powder after removal of hexane in vacuum. Yield 0.49 g (38%).

### CH₃CH₂CCl═N(4-C₆H₄CN)

To a solution of CH₃CH₂CONH(4-C₆H₄CN) in CH₂Cl₂ was added 1 eq. of PCl₅ and the reaction mixture was stirred for 1 h at room temperature. Solvent was then removed in vacuum and compound CH₃CH₂CCl═N(4-C₆H₄CN) was obtained.

¹H NMR (CH₂Cl₂): δ 7.50-7.52 (d, 2, NPhCN), 6.78-6.81 (d, 2, NPhCN), 2.64-2.71 (q, 2, CH₃CH₂CCl), 1.14-1.18 (t, 3, CH₃CH₂CCl).

### PhCH₂NHCO(4-C₆H₄CN)

To a solution of 4-cyanobenzoic acid (10 mmol, 1.66 g) in CH₂Cl₂ (50 mL) was added benzyl amine (10 mmol, 1.07 mL). The reaction mixture was stirred overnight at ambient temperature. The mixture was then filtered and the solvent of filtrate was removed in vacuum. Compound PhCH₂NHCO(4-C₆H₄CN) was obtained as a white powder. Yield 1.6 g (68%).

### PhCH₂N═CCl(4-C₆H₄CN)

To a solution of PhCH₂NHCO(4-C₆H₄CN) in CH₂Cl₂ (50 mL) was added 1 eq. of PCl₅ and the reaction mixture was stirred for 1 h at room temperature. Solvent was then removed in vacuum and compound PhCH₂N═CCl(4-C₆H₄CN) was obtained as a pale pink oil. Yield 1.3 g (75%).

¹H NMR (CH₂Cl₂): δ 7.57-7.60 (d, 2, NCPhCCl), 7.13-7.16 (d, 2, NCPhCCl), 6.76-6.86 (m, 4, NCH₂Ph), 6.68-6.73 (m, 1, NCH₂Ph), 4.35 (s, 2, NCH₂Ph).

### C₆H₁₁NHCO(4-C₆H₄CN)

To a solution of 4-cyanobenzoic acid (10 mmol, 1.66 g) in CH₂Cl₂ (50 mL) was added cyclohexyl amine (11 mmol, 1.09 mL) and triethylamine (22 mmol, 2.2 mL). The reaction mixture was stirred overnight at ambient temperature. The mixture was then filtered and the solvent of filtrate was removed in vacuum. Then the solid was extracted with Et₂O. Compound C₆H₁₁NHCO(4-C₆H₄CN) was obtained as a white powder after removal of Et₂O in vacuum. Yield 460 g (20%).

### C₆H₁₁N═CCl(4-C₆H₄CN)

To a solution of C₆H₁₁NHCO(4-C₆H₄CN) in CH₂Cl₂ (50 mL) was added 1 eq. of PCl₅ and the reaction mixture was stirred for 1 h at room temperature. Solvent was then removed in vacuum and compound C₆H₁₁N═CCl(4-C₆H₄CN) was obtained as a pale yellow powder. Yield 410 g (88%).

¹H NMR (CH₂Cl₂): δ 7.96-7.99 (d, 2, NCPhCCl), 7.56-7.59 (d, 2, NCPhCCl), 3.73-3.82 (tt, 1, CCl═NCH), 1.15-1.69 (m, 10, CCl═NCHC₅H₁₀).

### CH₃CH₂CONH(4-C₆H₄NO₂)

To a solution of 4-nitroaniline (7.5 mmol, 1.04 mg) in CH₂Cl₂ (30 mL) was added propionyl chloride (7.5 mmol, 0.70 mL) and the reaction mixture was stirred overnight at ambient temperature. The mixture was then filtered and the solvent of filtrate was removed in vacuum. The product was washed with hexane (10 mL). Compound N-(4-nitrophenyl)propionamide was obtained as a white powder after removal of hexane in vacuum. Yield 0.38 g (26%).

### CH₃CH₂CCl═N(4-C₆H₄NO₂)

To a solution of CH₃CH₂CONH(4-C₆H₄NO₂) in CH₂Cl₂ was added 1 eq. of PCl₅ and the reaction mixture was stirred for 1 h at room temperature. Solvent was then removed in vacuum and compound CH₃CH₂CCl═N(4-C₆H₄NO₂) was obtained.

¹H NMR (CH₂Cl₂): δ 8.06-8.09 (d, 2, NPhNO₂), 6.82-6.85 (d, 2, NPh NO₂), 2.66-2.73 (q, 2, CH₃CH₂CCl), 1.15-1.20 (t, 3, CH₃CH₂CCl).

### PhCONH(4-C₆H₄COCH₃)

To a solution of 1-(3-aminophenyl)ethanone (15 mmol, 2.03 g) in CH₂Cl₂ (30 mL) was added benzoyl chloride (15 mmol, 2.11 mL) and the reaction mixture was stirred overnight at ambient temperature. The mixture was then filtered and the solvent of filtrate was removed in vacuum. The product was washed with hexane (10 mL). Compound PhCONH(4-C₆H₄COCH₃) was obtained as a white powder after removal of hexane in vacuum. Yield 1.96 g (55%).

### PhCCl═N(4-C₆H₄COCH₃)

To a solution of PhCONH(4-C₆H₄COCH₃) in CH₂Cl₂ (15 mL) was added 1.1 eq. of distilled Cl₂SO and the reaction mixture was stirred overnight at 70° C. Solvent was then removed in vacuum and the product was distilled under vacuum. Compound PhCCl═N(4-C₆H₄COCH₃) was obtained as an orange-yellow oil. Yield 1.6 g (42%).

¹H NMR (CH₂Cl₂): δ 7.96-7.99 (d, 2, Ph(o)CCl═N), 7.58-7.61 (d, 1, NPhCOCH₃), 7.28-7.42 (m, 5, Ph(m, p)CCl═NPhCOCH₃), 7.00-7.04 (d, 1, NPhCOCH₃), 2.40 (s, 3, NPhCOCH₃).

### PhCONH(4-C₆H₄COOCH₂CH₃)

To a solution of ethyl-4-aminobenzoate (15 mmol, 2.43 g) in CH₂Cl₂ (30 mL) was added benzoyl chloride (15 mmol, 2.11 mL) and the reaction mixture was stirred overnight at ambient temperature. The mixture was then filtered and the solvent of filtrate was removed in vacuum. The product was washed with hexane (10 mL). Compound PhCONH(4-C₆H₄COOCH₂CH₃) was obtained as a white powder after removal of hexane in vacuum. Yield 2.21 g (55%).

### PhCCl═N(4-C₆H₄COOCH₂CH₃)

To a solution of PhCONH(4-C₆H₄COOCH₂CH₃) in CH₂Cl₂ was added 1 eq. of PCl₅ and the reaction mixture was stirred for 1 h at room temperature. Solvent was then removed in vacuum and compound PhCCl═N(4-C₆H₄COOCH₂CH₃) was obtained.

¹H NMR (CH₂Cl₂): δ 7.98-8.01 (d, 2, Ph(o)CCl═N), 7.90-7.93 (d, 2, NPhCOOCH₂CH₃), 7.40-7.45 (m, 1, Ph(p)CCl═N), 7.32-7.37 (m, 2, Ph(m)CCl═N), 6.86-6.89 (d, 2, NPhCOOCH₂CH₃), 4.14-4.21 (q, 2, NPhCOOCH₂CH₃), 1.18-1.23 (t, 3, NPhCOOCH₂CH₃).

### 4-(dimethylamino)-N-isopropyl benzamide

To a solution of 4-dimethylamino benzoyl chloride (10 mmol, 1.80 g) and Et₃N (10 mmol, 1.01 mL) in ether (100 mL) was slowly added isopropyl amine (12 mmol, 0.7 mL). The reaction mixture was stirred overnight at ambient temperature. The solvent was removed in vacuum and the product was washed with hexane (30 mL). Compound 4-(dimethylamino)-N-isopropyl benzamide was obtained as a white powder after removal of hexane in vacuum. Yield 1.21 g (60%).

### 4-Me₂NC₆H₄CCl═NCH(CH₃)₂

To a solution of 4-(dimethylamino)-N-isopropyl benzamide in CH₂Cl₂ was added 1 eq. of PCl₅ and the reaction mixture was stirred overnight at room temperature. Solvent was then removed in vacuum and compound 4-Me₂NC₆H₄CCl═NCH(CH₃)₂ was obtained as a yellow powder. Yield 1.33 g (98%).

¹H NMR (CH₂Cl₂): δ 8.12-8.15 (d, 2,4-Me₂Ph(3,5)CCl), 7.19-7.21 (d, 2,4-Me₂Ph(2,6)CCl), 4.20 (m, 1, Cl═NCH(CH₃)₂), 3.00 (s, 6,4-Me₂PhCCl), 1.28-1.30 (d, 6, Cl═NCH(CH₃)₂).

### 3-(trifluoromethyl)-N-isopropyl benzamide

To a solution of 3-trifluoromethyl benzoyl chloride (10 mmol, 2.0 mL) and Et₃N (10 mmol, 1.01 mL) in ether (100 mL) was slowly added isopropyl amine (12 mmol, 0.7 mL). The reaction mixture was stirred overnight at ambient temperature. The solvent was removed in vacuum and the product was washed with hexane (30 mL). Compound 3-(trifluoromethyl)-N-isopropyl benzamide was obtained as a white powder after removal of hexane in vacuum. Yield 1.94 g (85%).

### (3-CF₃C₆H₄)CCl═NCH(CH₃)₂

A solution of 3-(trifluoromethyl)-N-isopropyl benzamide in distilled Cl₂SO was refluxed for 2 hr. Solvent was then removed in vacuum and the product was dried under vacuum. Compound (3-CF₃C₆H₄)CCl═NHCH(CH₃)₂ was obtained as a white oil. Yield 1.90 g (90%).

¹H NMR (CH₂Cl₂): δ 8.12 (s, 1,3-CF₃Ph(2)CCl), 8.03-8.06 (d, 1,3-CF₃Ph(4)CCl), 7.56-7.59 (d, 1,3-CF₃Ph(6)CCl), 7.43 (m, 1,3-CF₃Ph(5)CCl), 4.02 (m, 1, Cl═NCH(CH₃)₂), 1.12-1.14 (d, 6, Cl═NCH(CH₃)₂).

### 4-chloro-N-isopropyl benzamide

To a solution of 4-chlorobenzoyl chloride (10 mmol, 1.75 mL) and Et₃N (10 mmol, 1.01 mL) in ether (100 mL) was slowly added isopropyl amine (12 mmol, 0.7 mL). The reaction mixture was stirred overnight at ambient temperature. The solvent was removed in vacuum and the product was washed with hexane (30 mL). Compound 4-chloro-N-isopropyl benzamide was obtained as a white powder after removal of hexane in vacuum. Yield 0.75 g (38%).

### (4-ClC₆H₄)CCl═NCH(CH₃)₂

To a solution of 4-chloro-N-isopropyl benzamide in CH₂Cl₂ was added 1 eq. of PCl₅ and the reaction mixture was stirred overnight at room temperature. Solvent was then removed in vacuum and compound (4-ClC₆H₄)CCl═NCH(CH₃)₂ was obtained as a yellow oil. Yield 0.60 g (79%).

¹H NMR (CH₂Cl₂): δ 7.77-7.80 (d, 2,4-ClPh(m)CCl), 7.23-7.25 (d, 2,4-ClPh(o)CCl), 3.98 (m, 1, Cl═NCH(CH₃)₂), 1.10-1.12 (d, 6, Cl═NCH(CH₃)₂).

### 4-CH₃OOCC₆H₄CONHCH(CH₃)₂

To a solution of methyl-4-(chlorocarbonyl)benzoate (7 mmol, 1.4 g) and Et₃N (8 mmol, 0.81 mL) in ether (100 mL) was slowly added isopropyl amine (8 mmol, 0.48 mL). The reaction mixture was stirred overnight at ambient temperature. The solvent was removed in vacuum and the product was washed with hexane (30 mL). Compound 4-CH₃OOCC₆H₄CONHCH(CH₃)₂ was obtained as a pale yellow powder after removal of hexane in vacuum. Yield 1.2 g (77%).

### 4-CH₃OOCC₆H₄CCl═NCH(CH₃)₂

To a solution of 4-CH₃OOCC₆H₄CONHCH(CH₃)₂ in CH₂Cl₂ was added 1 eq. of PCl₅ and the reaction mixture was stirred overnight at room temperature. Solvent was then removed in vacuum and compound 4-CH₃OOCC₆H₄CCl═NCH(CH₃)₂ was obtained as a light yellow powder. Yield 1.27 g (98%).

¹H NMR (CH₂Cl₂): δ 7.94-8.03 (dd, 4,4-CH₃OOCPhCCl), 4.18 (m, 1, Cl═NCH(CH₃)₂), 3.78 (s, 3,4-CH₃OOCPhCCl), 1.24-1.26 (d, 6, CNCH(CH₃)₂).

### II. NMR Scale Reduction of Imidoyl Chlorides

### (a) Representative Synthetic Procedure

In a representative procedure, to a solution of HSiMe₂Ph (145.0 μL, 1.04 mmol) and PhCCl═NCH₂Ph (150.0 mg, 0.69 mmol) in CD₂Cl₂ was added a solution of [Cp(Pr¹₃P)Ru(NCMe)₂]⁺[PF₆]⁻ (20 mg, 0.034 mmol) and t-BuCN (15 μL, 0.17 mmol) in CD₂Cl₂. The reaction was periodically monitored by NMR spectroscopy. PhCH═NCH₂Ph was obtained as a product.

### (b) NMR Spectroscopic Data of Products

### PhCH═NCH₂Ph

¹H NMR (CDCl₃): δ 4.88 (s, 2, PhCH═NCH₂Ph), 8.44 (s, 1, PhCH═NCH₂Ph), 7.39-7.86 (m, 10, PhCH═NCH₂Ph). ¹H-¹³C HSQC (CD₂Cl₂): δ 65.4 (s, PhCH═NCH₂Ph), 162.1 (s, PhCH═NCH₂Ph), 127.05-130.82 (s, PhCH═NCH₂Ph).

### ^{t}BuCH═NCH₂Ph

¹H NMR (CDCl₃): δ 4.61 (s, 2, (CH₃)₃CH═NCH₂Ph), 7.69 (s, 1, (CH₃)₃CH═NCH₂Ph), 1.15 (s, 1, (CH₃)₃CH═NCH₂Ph), 7.26-7.38 (m, 5, (CH₃)₃CH═NCH₂Ph). ¹H-¹³C HSQC (CD₂Cl₂): δ 64.5 (s, (CH₃)₃CH═NCH₂Ph), 27.0 (s, (CH₃)₃CH═NCH₂Ph), 173.5 (s, (CH₃)₃CH═NCH₂Ph), 126.8, 127.6, 128.4 (s, (CH₃)₃CH═NCH₂Ph).

### 4-MeOC₆H₄CH═NCH₂Ph

¹H NMR (CH₂Cl₂): δ 3.67 (s, 3, OCH₃), 4.59 (s, 2, CH₂), 6.67-6.70 (d, 2, CH₃OPh), 7.54-7.57 (d, 2, CH₃OPh), 6.97-7.04 (m, 3, CH₂Ph), 7.06-7.10 (m, 2, CH₂Ph).

### PhCH═N(4-C₆H₄COCH₃)

¹H NMR (CDCl₃): δ 2.66 (s, 3, PhCH═NPhCOCH₃), 8.52 (s, 1, PhCH═NPhCOCH₃), 7.27-7.96 (m, 9, PhCH═NPhCOCH₃). ¹H-¹³C HSQC (CD₂Cl₂): δ 26.8 (s, PhCH═NPhCOCH₃), 161.4 (s, PhCH═NPhCOCH₃).

### CH₃CH₂CH═N(4-C₆H₄COCH₃)

¹H NMR (CH₂Cl₂): δ 1.02-1.07 (t, 3, CH₃CH₂), 2.22-2.39 (m, 2, CH₃CH₂), 2.42 (s, 3, OCH₃), 7.17-7.21 (m, 2, NPhCOCH₃), 7.38-7.41 (m, 2, NPhCOCH₃) 7.74-7.77 (t, 1, CH).

### CH₃CH₂CH═N(4-C₆H₄COOCH₂CH₃)

¹H NMR (CH₂Cl₂): δ 1.01-1.06 (t, 3, CHCH₂CH₃), 1.21-1.24 (m, 3, OCH₂CH₃), 2.27-2.36 (m, 2, CHCH₂CH₃), 4.14-4.21 (m, 2, OCH₂CH₃), 6.83-6.85 (d, 2, Ph), 7.81-7.84 (d, 2, Ph), 7.69-7.72 (t, 1, CH).

### CH₃CH₂CH═NPh

¹H NMR (CH₂Cl₂): δ 1.01-1.06 (t, 3, CH₃CH₂), 2.25-2.34 (m, 2, CH₃CH₂), 6.82-6.85 (d, 2, NPh), 6.98-7.03 (t, 1, NPh), 7.38-7.41 (m, 2, NPh), 7.69-7.71 (t, 1, CH).

### 3-CF₃C₆H₄CH═NCH(CH₃)₂

¹H NMR (CH₂Cl₂): δ 1.28 (s, 3, CH₃CHCH₃), 1.30 (s, 3, CH₃CHCH₃), 3.61 (m, 1, CH₃CHCH₃), 8.07 (s, 1,3-CF₃Ph), 7.95 (d, 1,3-CF₃Ph), 7.56 (m, 1,3-CF₃Ph), 7.72 (m, 1,3-CF₃Ph), 8.38 (s, 1,3-CF₃PhCH═N).

### 4-ClC₆H₄CH═NCH(CH₃)₂

¹H NMR (CH₂Cl₂): δ 1.07 (s, 3, CH₃CHCH₃), 1.09 (s, 3, CH₃CHCH₃), 3.38 (m, 1, CH₃CHCH₃), 7.51 (d, 2,4-ClPh), 7.23 (d, 2,4-ClPh), 7.56 (m, 1,3-CF₃Ph), 7.72 (m, 1,3-CF₃Ph), 8.11 (s, 1,4-ClPhCH═N).

### III. Isolation of Imines

### (a) PhCH═NCH₂Ph

In a representative procedure, to a mixture solution of PhCH═NCH₂Ph and ClSiMe₂Ph in hexane was added 1 eq. of 2 M HCl in Et₂O. The precipitate was then dissolved in Et₂O and 1.2 eq. of Et₃N was added. The solution was filtered and the filtrate was dried under vacuum. Compound PhCH═NCH₂Ph was obtained as a yellow oil. Yield 0.42 g (43%).

¹H NMR (CDCl₃): δ 4.88 (s, 2, PhCH═NCH₂Ph), 8.44 (s, 1, PhCH═NCH₂Ph), 7.39-7.86 (m, 10, PhCH═NCH₂Ph). ¹H-¹³C HSQC (CD₂Cl₂): δ 65.4 (s, PhCH═NCH₂Ph), 162.1 (s, PhCH═NCH₂Ph), 127.05-130.82 (s, PhCH═NCH₂Ph). IR (neat): ∪ (C═N)=1025 cm⁻¹.

### (b) ^{t}BuCH═NCH₂Ph

To a mixture solution of (CH₃)₃CCH═NCH₂Ph and ClSiMe₂Ph in hexane was added 1 eq. of 2 M HCl in Et₂O. The precipitate was then dissolved in Et₂O and 2 eq. of Et₃N was added. The solution was filtered and the filtrate was dried under vacuum. Compound (CH₃)₃CCH═NCH₂Ph was obtained as a pale green oil. Yield 0.15 g (57%).

¹H NMR (CDCl₃): δ 4.61 (s, 2, (CH₃)₃CCH═NCH₂Ph), 7.69 (s, 1, (CH₃)₃CCH═NCH₂Ph), 1.15 (s, 1, (CH₃)₃CCH═NCH₂Ph), 7.26-7.38 (m, 5, (CH₃)₃CCH═NCH₂Ph). ¹H-¹³C HSQC (CD₂Cl₂): δ 64.5 (s, (CH₃)₃CCH═NCH₂Ph), 27.0 (s, (CH₃)₃CCH═NCH₂Ph), 173.5 (s, (CH₃)₃CCH═NCH₂Ph), 126.8, 127.6, 128.4 (s, (CH₃)₃CCH═NCH₂Ph). IR (neat): ∪ (C═N)=1029 cm⁻¹.

### (c) PhCH═N(4-C₆H₄COCH₃)

To a solution of PhCH═N(4-C₆H₄COCH₃) and ClSiMe₂Ph in hexane was added 1 eq. of 2 M HCl in Et₂O. The precipitate was then dissolved in Et₂O and 1.2 eq. of Et₃N was added. The solution was filtered and the filtrate was dried under vacuum. Compound PhCH═N(4-C₆H₄COCH₃) was obtained as a yellow oil. Yield 0.114 g (40%).

¹H NMR (CDCl₃): δ 2.66 (s, 3, PhCH═NPhCOCH₃), 8.52 (s, 1, PhCH═NPhCOCH₃), 7.27-7.96 (m, 9, PhCH═NPhCOCH₃). ¹H-¹³C HSQC (CD₂Cl₂): δ 26.8 (s, PhCH═NPhCOCH₃), 161.4 (s, PhCH═NPhCOCH₃). IR (neat): ∪ (C═N)=1074 cm⁻¹.

### IV. Isolation of Aldehydes

### (a) 3-CF₃C₆H₄CCl═NCHMe₂

After the reaction was completed, the catalyst was removed by extracting with hexane. Then the mixture of 3-CF₃C₆H₄CH═NCHMe₂ and ClSiMe₂Ph was hydrolysed by adding H₂O/HCl. The 3-CF₃C₆H₄CHO and PhMe₂SiOSiMe₂Ph were then extracted with CH₂Cl₂ and the solution was dried over MgSO₄. The 3-CF₃C₆H₄CHO was isolated by chromatography over silica using 15:1 hexane:ethyl acetate as eluent to afford the product as a white oil. (89 mg, 64% yield).

### 3-CF₃C₆H₄CHO

¹H NMR (CH₂Cl₂): δ 10.02 (s, 1, PhCHO), 8.10 (s, 1, CF₃Ph(2)), 8.03-8.05 (d, 1, CF₃Ph(4)), 7.84-7.87 (d, 1, CF₃Ph(6)), 7.64-7.69 (t, 1, CF₃Ph(5)). ¹⁹F NMR (CDCl₃): δ −62.94 (s, 1,3-CF₃PhCHO). ¹H-¹³C HSQC (CDCl₃): δ 186.3 (PhCHO) 132.4 (CF₃Ph(4)), 131.0 (CF₃Ph(6)), 129.7 (CF₃Ph(5)), 126.5 (CF₃Ph(2)).

### (b) 4-ClC₆H₄CCl═NCHMe₂

After the reaction was completed, the catalyst was removed by extracting with hexane. Then the mixture of 4-ClC₆H₄CH═NCHMe₂ and ClSiMe₂Ph was hydrolysed by adding H₂O/HCl. The 4-ClC₆H₄CHO and PhMe₂SiOSiMe₂Ph were then extracted with CH₂Cl₂ and the solution was dried over MgSO₄. The 4-ClC₆H₄CHO was isolated by chromatography over silica using 20:1 hexane:ethyl acetate as eluent to afford the product as a white solid. (71 mg, 51% yield).

### 4-ClC₆H₄CHO

¹H NMR (CH₂Cl₂): δ 9.86 (s, 1, PhCHO), 7.70-7.73 (d, 2, ClPh(m)), 7.41-7.44 (d, 2, ClPh(m)). ¹³C NMR (CH₂Cl₂): δ 190.4 (PhCHO) 140.5 (4-ClPh(4)), 134.7 (4-ClPh(l)), 130.7 (4-ClPh(3, 5)), 129.2 (4-ClPh(2,6)).

### (c) 4-CH₃OOCPhCCl═NCHMe₂

After the reaction was completed, the catalyst was removed by extracting with hexane. Then, the mixture of 4-CH₃OOCPhCH═NCHMe₂ and ClSiMe₂Ph was hydrolysed by adding H₂O/HCl. The 4-CH₃OOCPhCHO and PhMe₂SiOSiMe₂Ph were then extracted with CH₂Cl₂ and the solution was dried over MgSO₄. The 4-CH₃OOCPhCHO was isolated by chromatography over silica using 15:1 hexane:ethyl acetate as eluent to afford the product aldehyde as a white powder (75 mg, 46% yield).

### 4-CH₃OOCPhCHO

¹H NMR (CH₂Cl₂): δ 9.96 (s, 1, PhCHO), 8.05-8.07 (d, 2,4-CH₃OOCPh), 7.81-7.84 (d, 2,4-CH₃OOCPh), 3.81 (s, 3,4-CH₃OOCPh). ¹H-¹³C HSQC (CH₂Cl₂): δ 191.5 (PhCHO), 129.9 (4-CH₃OOCPh), 129.1 (4-CH₃OCPh), 52.3 (4-CH₃OOCPh).

### V. Discussion

Complex [Cp(Prⁱ₃P)Ru(NCMe)₂]⁺[PF₆]⁻ was used as a catalyst for the reduction of imidoyl chlorides to the corresponding imines (Tables 3 and 4) and aldehydes (Table 5) in the presence of HSiMe₂Ph as the reducing agent. The reaction was observed to proceed in CH₂Cl₂ and t-BuCN (20-25 mol %) was added to inhibit possible decomposition of the catalyst.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

¹ a) M. Hudlicky, Reductions in Organic Chemistry, ACS monograph No. 188, American Chemical Society, Washington D.C., 1996; b) R. C. Larock, Comprehensive Organic Transformations: a Guide to Functional Group Preparation, 2nd ed., Wiley-VCH, New York, 1999; c) Comprehensive Organic Chemistry, V.3, Part 9 (Eds: D. Barton, W. D. Ollis), Pergamon, Oxford, 1979.
² Amides: a) S. Das, D. Addis, S. Zhou, K. Junge, M. Beller, J. Am. Chem. Soc. 2010, 132, 1770; b) S. Zhou, K. Junge, D. Addis, S. Das, M. Beller, Angew. Chem. Int. Ed. 2009, 48, 9507; c) Y. Sunada, H. Kawakami, T. Imaoka, Y. Motoyama, H. Nagashima, Angew. Chem. Int. Ed. 2009, 48, 9511; d) S. Hanada, E. Tsutsumi, Y. Motoyama, H. Nagashima, J. Am. Chem. Soc. 2009, 131, 15032; e) S. Hanada, T. Ishida, Y. Motoyama, H. Nagashima, J. Org. Chem. 2007, 72, 7551; f) K. Selvakumar, K. Rangareddy, J. F. Harrod, Can. J. Chem. 2004, 82, 1244; g) K. Selvakumar, J. F. Harrod, Angew. Chem. Int. Ed. 2001, 40, 2129; h) K. Rangareddy, K. Selvakumar, J. F. Harrod, J. Org. Chem. 2004, 69, 6843; i) M. Igarashi, T. Fuchikami, Tetrahedron Lett. 2001, 42, 1945; j) R. Kuwano, M. Takahashi, Y. Ito, Tetrahedron Lett. 1998, 39, 1017; k) N. Sakai, K. Fujii, T. Konakahara, Tetrahedron Lett. 2008, 49, 6873; l) A. C. Fernandes, C. C. Romao, J. Mol. Catal. A: Chem. 2007, 272, 60; m) H. Sasakuma, Y. Motoyama, H. Nagashima, Chem. Commun. 2007, 46, 4916; n) Y. Motoyama, K. Mitsui, T. Ishida, H. Nagashima, J. Am. Chem. Soc. 2005, 127, 13150; o) S. Hanada, Y. Motoyama, H. Nagashima, Tetrahedron Lett. 2006, 47, 6173; p) S. Bower, K. A. Kreutzer, S. L. Buchwald, Angew. Chem. Int. Ed. Engl. 1996, 35, 1515.
³ Esters: a) S. C. Berc, K. A. Kreutzer, S. L. Buchwald, J. Am. Chem. Soc. 1991, 113, 5093; b) S. C. Berc, S. L. Buchwald, J. Org. Chem. 1992, 57, 3751; c) K. J. Barr, S. C. Berk, S. L. Buchwald, J. Org. Chem. 1994, 59, 4323; d) S. W. Breeden, N.J. Lawrence, Synlett 1994, 833; e) Z. Mao, B. T. Gregg, A. R. Cutler, J. Am. Chem. Soc. 1995, 117, 10139; f) M. Igarashi, R. Mizuno, T. Fuchikami, Tetrahedron Lett. 2001, 42, 2149; g) A. C. Fernandes, C. C. Romao, J. Mol. Catal. A: Chem. 2006, 253, 96; h) N. Sakai, T. Moriya, T. Konakahara, J. Org. Chem. 2007, 72, 5920; i) T. Ohta, M. Kamiya, M. Nobutomo, K. Kusui, I. Furukawa, Bull. Chem. Soc. Jpn. 2005, 78, 1856; j) T. Ohta, M. Kamiya, K. Kusui, T. Michibata, M. Nobutomo, I. Furukawa, Tetrahedron Lett. 1999, 40, 6963; k) K. Matsubara, T. Iura, T. Maki, H. Nagashima, J. Org. Chem. 2002, 67, 4985.
⁴ Nitriles: a) R. Calas, E. Frainnet, A. Bazouin, Compt. Rend. 1961, 252, 420; b) T. Fuchigami, I. Igarashi, Japanese Patent Application JP11228579, 1999; c) A. M. Caporusso, N. Panziera, P. Pertici, E. Pitzalis, P. Salvadori, G. Vitulli, G. Martra, J. Mol. Cat. A: Chem. 1999, 150, 275; d) T. Murai, T. Sakane, S. Kato, J. Org. Chem. 1990, 55, 449; e) T. Murai, T. Sakane, S. Kato, Tetrahedron Lett. 1985, 26, 545.
⁵ Nitriles: a) A. Y. Khalimon, R. Simionescu, L. G. Kuzmina, J. A. K. Howard, G. I. Nikonov, Angew. Chem. Int. Ed. 2008, 47, 7704; b) E. Peterson, A. Y. Khalimon, R. Simionescu, L. G. Kuzmina, J. A. K. Howard, G. I. Nikonov, J. Am. Chem. Soc. 2009, 131, 908; c) D. V. Gutsulyak, G. I. Nikonov, Angew. Chem. Int. Ed. 2010, 49, 7553.
⁶ For related catalytic C-0 bond cleavage by silanes in alkyl ethers and epoxides, see: a) S. Park, M. Brookhart, Chem. Commun. 2011, 47, 3643; b) J. Yang, P. S. White, M. Brookhart, J. Am. Chem. Soc. 2008, 130, 17509.
⁷ a) H. C. Brown, R. F. McFarlin, J. Am. Chem. Soc. 1956, 78, 252; b) H. C. Brown, B. C. Subba Rao, J. Am. Chem. Soc. 1958, 80, 5377; c) H. C. Brown, S. Krishnamurthy, Tetrahedron 1979, 35, 567; d) J. S. Cha, H. C. Brown, J. Org. Chem. 1993, 58, 4732; e) M. A. Delasheras, J. J. Vaquero, J. L. Garcianavio, J. Alvarezbuilla, Tetrahedron Lett. 1995, 36, 455.
⁸ a) P. Four, F. Guibe, J. Org. Chem. 1981, 46, 4439; b) C. Malanga, S. Mannucci, L. Lardicci, Tetrahedron Lett. 1997, 38, 8093; c) K. Inoue, M. Yasuda, I. Shibata, A. Baba, Tetrahedron Lett. 2000, 41, 113; d) P. Le Ménez, A. Hamze, O. Provot, J.-D. Brion, M. Alami, Synlett 2010, 1101.
⁹ a) T. N. Sorrell, P. S. Pearlman, J. Org. Chem. 1980, 45, 3449; b) J. S. Cha, Org. Prep. Proced. Int. 1989, 21, 451; c) J. C. Leblanc, C. Moïse, J. Tirouflet, J. Organomet. Chem. 1985, 292, 225; d) P. L. Gaus, S. C. Kao, K. Youngdahl, M. Y. Darensbourg, J. Am. Chem. Soc. 1985, 107, 2203.
¹⁰ a) X. Jia, X. Liu, J. Li, P. Zhao, Y. Zhang, Tetrahedron Lett. 2007, 48, 971; b) H. Maeda, T. Maki, H. Ohmori, Tetrahedron Lett. 1995, 36, 2247.
¹¹ Stoichiometric reduction by hypervalent silicon hydrides has been reported: R. J. P. Corriu, G. F. Lanneau, M. Perrot, Tetrahedron Lett. 1988, 29, 1271.
¹² For the use of noble metal catalysis, see: a) J. W. Jenkins, H. W. Post, J. Org. Chem. 1950, 15, 556; b) J. D. Citron, J. Org. Chem. 1969, 34, 1977; c) B. Courtis, S. P. Dent, C. Eaborn, A. Pidcock, J. Chem. Soc. Dalton 1975, 2460; d) S. P. Dent, C. Eaborn, A. Pidcock, J. Chem. Soc. Dalton 1975, 2646; e) K. Lee, R. E. Maleczka Jr., Org. Lett. 2006, 8, 1887.
¹³ Related Pd-catalyzed reduction of aromatic acyl fluorides is also known: R. Braden, T. Himmler, J. Organomet. Chem. 1989, 367, C12.
¹⁴ D. V. Gutsulyak, S. F. Vyboishchikov, G. I. Nikonov, J. Am. Chem. Soc. 2010, 132, 5950.
¹⁵ D. V. Gutsulyak, G. I. Nikonov, Angew. Chem. Int. Ed. 2011, 50, 1384.
¹⁶ See, for example: C. A. Reed, Carborane acids. New “strong yet gentle” acids for organic and inorganic chemistry. Chem. Commun. 2005, 1669-1677.
¹⁷ A. L. Osipov, D. V. Gutsulyak, L. G. Kuzmina, J. A. K. Howard, D. A. Lemenovskii, G. Süss-Fink, G. I. Nikonov, J. Organomet. Chem., 2007, 692, 5081.
¹⁸ M. H. Novice, H. R. Seikaly, A. D. Seiz, T. T. Tidwell, J. Am. Chem. Soc. 1980, 102, 5835.

TABLE 1
Catalytic hydrosilylation of acid chlorides with HSiMe₂Ph

| Entry | Substrate | Conversion^{[a]} (time) | Products (yield)^{[a]} |
|---|---|---|---|
| 1 | C₆H₅COCl | 100% (1 h) | C₆H₅CHO (~100%) |
| 2 | CH₃COCl | >90% (2 h) | CH₃CHO (70%) |
| | | | CH₂═CHOSiMe₂Ph (20%) |
| 3 | CH₃CH₂COCl | 100% (1 h) | CH₃CH₂CHO (~100%) |
| 4 | (CH₃)₃CCOCl | 100% (4 h) | (CH₃)₃CCHO (~100%) |
| 5 | ClCH₂COCl | 100%^{[b]} (1 h) | ClCH₂CHO (90%) |
| 6 | CH₃CHClCOCl | 100% (24 h) | CH₃CHClCHO (95%) |
| 7 | ClCH₂CH₂COCl | 100% (5 h) | ClCH₂CH₂CHO (80%) |
| | | | ClCH═CHOSiMe₂Ph (20%) |
| 8 | BrCH₂CH₂COCl | 80% (24 h) | BrCH₂CH₂CHO (traces) |
| 9 | PhCH═CHCOCl | 70%^{[b]} (18 h) | PhCH═CHCHO (18%) |
| | | | PhCH₂CH═CHOSiMe₂Ph (82%) |
| 10 | 4-BrC₆H₄COCl | 100% (3 h) | 4-BrC₆H₄CHO (~100%) |
| 11 | 4-O₂NC₆H₄COCl | 90%^{[b]} (5 h) | 4-O₂NC₆H₄CHO (85%) |
| 12 | 4-MeOC₆H₄COCl | >90% (24 h) | 4-MeOC₆H₄CHO (83%) |
| | | | 4-MeOC₆H₄CH₂OSiMe₂Ph (17%) |
| 13 | 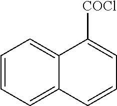 [SMILES: O=C(Cl)c1cccc2ccccc12] | 100% (20 h) | 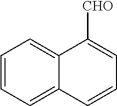 [SMILES: O=Cc1cccc2ccccc12] (95%) |
| 14 | 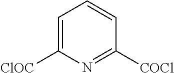 | 100%^{[b,c]} (3 h) | 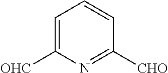 (80%) |
| 15 | 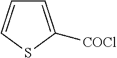 | >97% (24 h) | 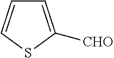 (97%) |
| 16 | 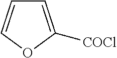 | 100% (24 h) | 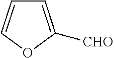 (~100%) |
| 17 | 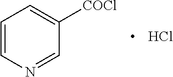 | 100%^{[b,c,d]} (24 h) | 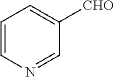 (traces) |
| 18 | EtOOC—COCl | 100%^{[b,e]} (20 h) | EtOOCCHO (65%) |

^{[a]}Based on ¹H NMR data.
^{[b]}2 equiv. of CH₃CN were added instead of t-BuCN.
^{[c]}2.5 equiv. of HSiMe₂Ph were added.
^{[d]}Conversion of silane is given.
^{[e]}Reaction in chloroform.

TABLE 2
Reduction of p-BrC₆H₄COCl with
HSiMe₂Ph in the presence of other substrates.

| Entry | Substrate 1 | Substrate 2 | Conversion ^{[a]} |
|---|---|---|---|
| 1 2 3 4 | 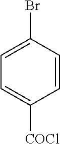 | CH₃(CH₂)₃CH═CH₂ AcOEt EtC≡CEt PhC≡CH | Substrate 1:~100%; Substrate 2:0% Substrate 1:90%; Substrate 2:0% Substrate 1:50%; Substrate 2:50% Substrate 1:50% Substrate 2:5% |

^{[a]} Based on ¹H NMR data.

TABLE 3
NMR scale reduction of imidoyl chlorides^{[a]}

| No | Imidoyl Chloride | Product | Time | | Conversion |
|---|---|---|---|---|---|
| 1 | PhCCl═NCH₂Ph | PhCH═NCH₂Ph | 15 | m | 100% |
| 2 | 4-MeOPhCCl═NCH₂Ph | 4-MeOPhCH═NCH₂Ph | 14 | h | 90% |
| 3 | t-BuCCl═NCH₂Ph | t-BuCCH═NCH₂Ph | 14 | h | 95% |
| 4 | CH₃CH₂CCl═NPh | CH₃CH₂CH═NPh | 30 | m | 80% |
| 5 | CH₃CH₂CCl═NPhCOCH₃ | CH₃CH₂CH═NPhCOCH₃ | 90 | m | 93% |
| 6 | CH₃CH₂CCl═NPhCOOCH₂CH₃ | CH₃CH₂CH═NPhCOOCH₂CH₃ | 3 | h | 86% |
| 7 | 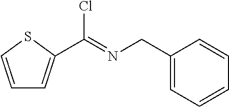 [SMILES: Cl/C(=N\Cc1ccccc1)c1cccs1] | Mixture of products | 13 | h | 90% |
| 8 | 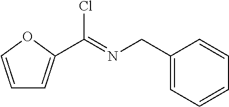 [SMILES: Cl/C(=N\Cc1ccccc1)c1ccco1] | — | NR^{[b]} | | — |
| 9 | 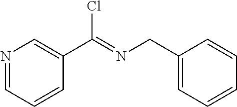 [SMILES: Cl/C(=N\Cc1ccccc1)c1cccnc1] | Mixture of products | 24 | h | 100% |
| 10 | PhCH═CHCCl═NCH₂Ph | Mixture of products | 12 | h | 100% |
| 11 | CH₃CH₂CCl═NPhCN | Hydrosilylation of nitrile | 2.5 | h | 30% |
| 12 | PhCH₂N═CClPhCN | Hydrosilylation of nitrile | 40 | m | 90% |
| 13 | C₆H₁₁N═CClPhCN | Hydrosilylation of nitrite | 3 | h | 100% |
| 14 | CH₃CH₂CCl═NPhNO₂ | — | NR^{[b]} | | — |
| 15 | PhCCl═NPhCOCH₃ | PhCH═NPhCOCH₃ | 20 | h | 65% |

| | | | | |
|---|---|---|---|---|
| 16 | PhCCl═NPhCOOCH₂CH₃ | — | NR^{[b]} | — |

^{[a]}Catalyst [Cp(Prⁱ₃P)Ru(NCMe)₂]⁺[PF₆]⁻ (5 mol %), t-BuCN (25 mol %), substrate (0.05-0.1 mmol), HSiMe₂Ph (1.1 eq) in CH₂Cl₂ (0.5 mL) at room temperature.
^{[b]}No reaction.

TABLE 4
Preparative scale reduction of imidoyl chlorides^{[a]}

| No | lmidoyl Chloride | Time | Conversion | Product |
|---|---|---|---|---|
| 1 | 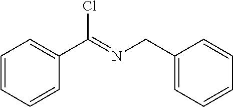 [SMILES: Cl/C(=N\Cc1ccccc1)c1ccccc1] | 50 m | 100% (43%)^{[b]} | 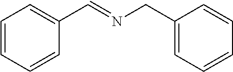 [SMILES: C(=N\Cc1ccccc1)/c1ccccc1] |
| 2 | 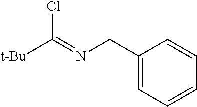 [SMILES: CC(C)(C)/C(Cl)=N/Cc1ccccc1] | 25 m | 100% (57%)^{[b]} | 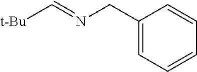 [SMILES: CC(C)(C)/C=N/Cc1ccccc1] |
| 3 | 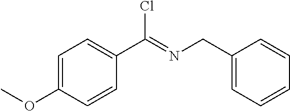 [SMILES: COc1ccc(/C(Cl)=N/Cc2ccccc2)cc1] | 5 h | 100% | The corresponding imine was obtained but a mixture of products was obtained after isolation. |
| 4 | 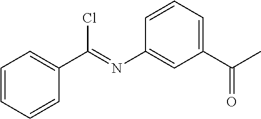 [SMILES: CC(=O)c1cccc(/N=C(\Cl)c2ccccc2)c1] | 7 d | 98% (40%)^{[b]} | 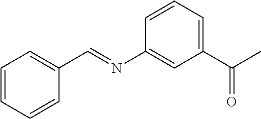 [SMILES: CC(=O)c1cccc(/N=C/c2ccccc2)c1] |
| 5 | 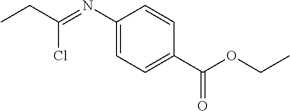 [SMILES: CCOC(=O)c1ccc(/N=C(\Cl)CC)cc1] | 3 h | 90% | The corresponding imine was obtained but a mixture of products was obtained after isolation. |

^{[a]}Catalyst [Cp(Prⁱ₃P)Ru(NCMe)₂]⁺[PF₆]⁻ (5 mol %), t-BuCN (20 mol %), substrate (1.2-6.0 mmol), HSiMe₂Ph (1 eq) in CH₂Cl₂ (12 mL) at room temperature.
^{[b]}Isolated yield.

TABLE 5
Reduction of imidoyl chlorides and isolation of aldehydes^{[a]}

| No | Imidoyl Chloride | Time | Conversion | Product^{[b]} | Yield |
|---|---|---|---|---|---|
| 1 | 3-CF₃PhCCl═NCHMe₂ | 70 m | 100% | 3-CF₃PhCH═NCHMe₂ | 100% |
| | | | | | (64%)^{[c]} |
| 2 | 4-ClPhCCl═NCHMe₂ | 50 m | 100% | Mixture of | 85% |
| | | | | 4-ClPhCH═NCHMe₂ and | (51%)^{[c]} |
| | | | | 4-ClPhCHO | |
| 3 | 4-CH₃OOCPhCCl═NCHMe₂ | 30 m | 100% | Mixture of | 97% |
| | | | | 4-CH₃OOCPhCH═NCHMe₂ | (46%)^{[c]} |
| | | | | and 4-CH₃OOCPhCHO | |

^{[a]}Catalyst [CpRu(ⁱPr₃P)(CH₃CN)₂]⁺[PF₆]⁻ (5 mol %), t-BuCN (20 mol %), substrate (1.0-1.6 mmol), HSiMe₂Ph (1 eq) in CH₂Cl₂ (12 mL) at room temperature.
^{[b]}Product of catalytic reduction reaction.
^{[c]}Isolated yield of aldehyde after hydrolysis.

1. A method for the catalytic reduction of a compound selected from an acid chloride and an imidoyl chloride, the method comprising reacting the compound with a silane reducing agent in the presence of a catalyst of Formula I: 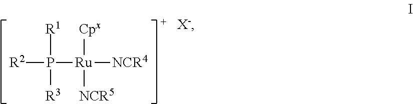
wherein
Cp^{x} is unsubstituted η⁵-cyclopentadienyl or η⁵-cyclopentadienyl substituted with 1 to 5 methyl groups;
R¹, R² and R³ are each independently selected from C₁₋₆alkyl and C₆₋₁₀aryl;
R⁴ and R⁵ are each independently C₁₋₄alkyl; and
X⁻ is a counteranion.

2. The method of claim 1, wherein Cp^{x} is unsubstituted η⁵-cyclopentadienyl.

3. The method of claim 1, wherein the silane reducing agent is selected from dimethylphenylsilane, triethylsilane, methylphenylsilane and triphenylsilane.

4. The method of claim 3, wherein the silane reducing agent is dimethylphenylsilane.

5. The method of claim 1, wherein R¹, R² and R³ are each isopropyl.

6. The method of claim 1, wherein R⁴ and R⁵ are each CH₃.

7. The method of claim 1, wherein X⁻ is selected from [PF₆]⁻, [ClO₄⁻], [B[3,5-(CF₃)₂C₆H₃]₄]⁻, [B(C₆F₅)₄]⁻, [Al(OC(CF₃)₃)₄]⁻, a carborane-based counteranion and a non-nucleophilic amide counteranion.

8. The method of claim 1, wherein the catalyst of Formula I is [Cp(Pr¹₃P)Ru(NCMe)₂]⁺[PF₆]⁻.

9. The method of claim 1, wherein the compound is an acid chloride having the structure: 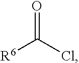
wherein R⁶ is
C₁₋₁₀alkyl, optionally substituted with chloro;
C₆₋₁₄aryl, optionally substituted with halo, nitro or C₁₋₄alkoxy,
heteroaryl; or
—C(O)OR⁷, wherein R⁷ is C₁₋₆alkyl.

10. The method of claim 1, wherein the compound is an acid chloride having the structure: 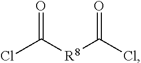
wherein R⁸ is C₁₋₁₀alkylene, C₆₋₁₄arylene or heteroarylene.

11. The method of claim 1, wherein the compound is an imidoyl chloride having the structure: 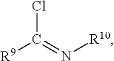
wherein
R⁹ is C₁₋₁₀alkyl or is C₆₋₁₄aryl, optionally substituted with C₁₋₄alkoxy; and
R¹⁰ is C₁₋₆alkyleneC₆₋₁₄aryl or is C₆₋₁₄aryl, optionally substituted with a —C(O)R¹¹ group or a —C(O)OR¹² group, wherein R¹¹ and R¹² are, independently, C₁₋₆alkyl.

12. The method of claim 1, wherein the catalyst is present in an amount of from about 0.2 mol % to about 20 mol %, based on the amount of the compound being reduced.

13. The method of claim 1, wherein the reaction of the compound with the silane reducing agent is carried out in the presence of at least one solvent.

14. The method of claim 13, wherein the solvent is selected from chloroform, dichloromethane, acetone and acetonitrile.

15. The method of claim 13, wherein the solvent is selected from chloroform, dichloromethane and acetone.

16. The method of claim 15, wherein the reaction of the compound with the silane reducing agent is further carried out in the presence of a C₁₋₆alkyl cyanide.

17. The method of claim 16, wherein the C₁₋₆alkyl cyanide is tBuCN or CH₃CN.

18. The method of claim 17, wherein the C₁₋₆alkyl cyanide is present in an amount of from about 5 mol % to about 250 mol %, based on the amount of the compound being reduced.

19. The method of claim 1, wherein the silane reducing agent is present in an amount of about 1 equivalent to about 2 equivalents, based on the amount of a functional group being reduced.

20. The method of claim 1, wherein the catalyst of Formula I is generated in situ from the reaction of a catalyst precursor of Formula II: 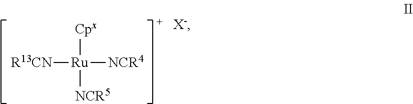
wherein
Cp^{x} is unsubstituted η⁵-cyclopentadienyl or η⁵-cyclopentadienyl substituted with 1 to 5 methyl groups;
R⁴, R⁵ and R¹³ are each independently C₁₋₄alkyl; and
X⁻ is a counteranion,
with a phosphine of Formula III: 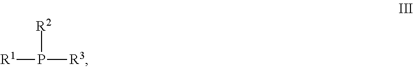
wherein
R¹, R² and R³ are each independently selected from C₁₋₆alkyl and C₆₋₁₀aryl.

21. The method of claim 1, wherein the reaction of the compound with the silane reducing agent in the presence of the catalyst of Formula I is carried out at a temperature of about 20° C. to about 25° C.
